(19) United States
(12) Patent Application Publication
(10) Pub. No.: US 2013/0005802 A1
(43) Pub. Date: Jan. 3, 2013

(54) TREATMENT OF WNT/FRIZZLED-RELATED DISEASES

(76) Inventors: Wei Chen, Chapel Hill, NC (US); Minyong Chen, Durham, NC (US); H. Kim Lyerly, Chapel Hill, NC (US); Lawrence S. Barak, Durham, NC (US); Robert Mook, Chapel Hill, NC (US); Takuya Osada, Chapel Hill, NC (US); Michael A. Morse, Raleigh, NC (US)

(21) Appl. No.: 13/497,364

(22) PCT Filed: Sep. 21, 2010
(86) PCT No.: PCT/US10/49693

§ 371 Date: Sep. 18, 2012

Related U.S. Application Data
(60) Provisional application No. 61/244,399, filed on Sep. 21, 2009.

(51) Int. Cl. A61K 31/167; G01N 33/574; A61P 9/00; C12Q 1/68; A61K 31/277; A61K 31/18

(52) U.S. Cl. 514/521; 514/622; 514/617; 514/604; 514/619; 435/6.11; 435/7.23; 435/7.1; 435/6.12; 435/7.92; 436/501

(57) ABSTRACT

Methods of treating Wnt/Frizzled-related diseases, comprising administering niclosamide compounds are provided. Methods of predicting whether a disease will respond to treatment with a niclosamide compound are also provided.

### CROSS-REFERENCE TO RELATED APPLICATIONS

This application claims the benefit of priority to U.S. Provisional Patent Application No. 61/244,399, filed Sep. 21, 2009, which is incorporated herein by reference in its entirety.

### STATEMENT REGARDING FEDERALLY SPONSORED RESEARCH

This invention was made with government support under Grant No. 5RO1 CA113656-03, awarded by the National Institutes of Health. The government has certain rights in the invention.

### SEQUENCE LISTING

The sequence listing is filed with the application in electronic format only and is incorporated by reference herein. The sequence listing text file “B2437638.txt” was created on Sep. 21, 2010 and is 33,888 bytes in size.

### INTRODUCTION

The Wnt signaling pathway plays fundamental roles throughout the life cycle of an organism—in the developing embryo by directing tissue patterning, and in the mature organism by maintaining tissue homeostasis and is involved in cancer. Wnt ligands are secreted glycoproteins that exist in multiple forms. Humans express 19 different Wnt subtypes that are agonists for at least 10 different seven transmembrane Frizzled receptors. Frizzled receptor is a cell surface receptor having seven transmembrane domains. The binding of Wnt to the Frizzled receptor activates cytosolic Dishevelled proteins, which leads to Frizzled receptor internalization. Downstream signaling events produced as a consequence of Wnt binding include the stabilization of cytosolic β-catenin by preventing GSK3β phosphorylation and translocation of the stabilized β-catenin to the nucleus followed by the activation of the transcription factor LEF/TCF.

Thus, Wnt proteins bind to Frizzled receptors to mediate the developmental, morphogenetic, and tissue-regenerative effects of Wnt signaling. Dysregulated Wnt signaling is associated with many cancers. Accordingly, the various proteins involved in the regulation of the Wnt signaling cascade, including accessible plasma membrane receptors like Frizzled, provide targets for therapeutic intervention. Nevertheless, there are currently no FDA-approved drugs or clinical candidates that modulate Wnt-mediated receptor trafficking, and subsequent Wnt signaling.

### SUMMARY

In an aspect the disclosure provides a method of predicting responsiveness of a cancer cell to treatment with a niclosamide compound, comprising determining the level of at least one protein in the cancer cell, wherein the protein is involved in the Wnt/Frizzled signaling pathway; and comparing the level of the protein to a standard level, wherein a difference between the levels of the protein indicates that the cancer cell is responsive to treatment with the niclosamide compound. The level of the protein may be determined by determining the gene expression level of the protein. The protein may be selected from the group consisting of cytosolic β-catenin, a Wnt protein, Frizzled, and Dishevelled.

In an aspect the disclosure provides a method of identifying a subject with a Wnt/Frizzled-related disease, comprising determining the level of at least one protein in a sample from the subject, wherein the protein is involved in the Wnt/Frizzled signaling pathway; and comparing the level of the protein to a standard level, wherein a difference between the levels of the protein is indicative of a subject having a Wnt/Frizzled-related disease. The level of the protein may be determined by determining the gene expression level of the protein. The protein may be selected from the group consisting of cytosolic β-catenin, a Wnt protein, Frizzled, and Dishevelled.

In an aspect the disclosure provides a method of treating a subject with a Wnt/Frizzled-related disease, comprising determining the level of at least one protein in a sample from the subject, wherein the protein is involved in the Wnt/Frizzled signaling pathway; comparing the level of the protein to a standard level, wherein a difference between the levels of the protein is indicative of a subject having a Wnt/Frizzled-related disease; and administering to the subject a niclosamide compound in an amount effective to treat the disease, wherein the Wnt/Frizzled-related disease is not a neoplasm. In certain aspects, the Wnt/Frizzled-related disease is a cardiovascular disease. The level of the protein may be determined by determining the gene expression level of the protein. The protein may be selected from the group consisting of cytosolic β-catenin, a Wnt protein, Frizzled, and Dishevelled.

In an aspect the disclosure provides a method of detecting a neoplasm in a subject, comprising determining the level of protein in a sample from the subject, wherein the protein is involved in the Wnt/Frizzled signaling pathway; and comparing the level of the at least one protein to a standard level, wherein a difference between the levels of the protein is indicative of the subject having a neoplasm. The level of the protein may be determined by determining the gene expression level of the protein. The protein may be selected from the group consisting of cytosolic β-catenin, a Wnt protein, Frizzled, and Dishevelled. The neoplasm may be a cancer selected from colon cancer, melanoma, hepatocellular carcinoma, leukemia, ovarian cancer, prostate cancer, lung cancer, brain tumor, and breast cancer.

In an aspect the disclosure provides a method of treating a Wnt/Frizzled-related disease in a subject in need of such treatment, the method comprising administering to the subject an effective amount of a niclosamide compound, or pharmaceutically acceptable salt thereof, of Formula I:

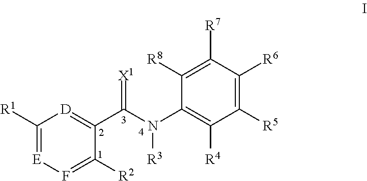

wherein
D is N or CR⁹;
E is N or CR¹⁰;
F is N or CR¹¹;
R¹ is H, halide, OR¹², SR¹³NR¹⁴R¹⁵, or described by one of the formulas:

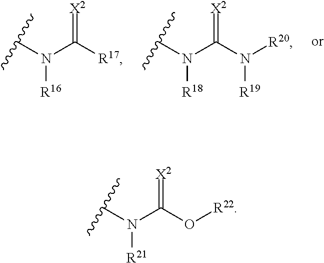

R² is H, OH, or OR¹²;
R³ is H, C₁₋₇ alkyl, C₂₋₇ alkenyl, C₂₋₇ alkynyl, C₂₋₆ heterocyclyl, C₆₋₁₂ aryl, C₇₋₁₄ alkylaryl, C₃₋₁₀ alkylheterocyclyl, or C₁₋₇ heteroalkyl;
or R² and R³ combine to form a six-membered ring in which position 1 is connected to position 4 by one of the groups:

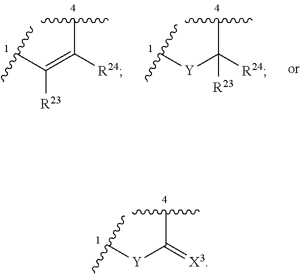

R⁴ and R⁸ are each, independently, selected from H, halide, CF₃, OR²⁸, C₁₋₇ alkyl, C₂₋₇ alkenyl, C₂₋₇ alkynyl, C₂₋₆ heterocyclyl, C₇₋₁₄ alkaryl, C₃₋₁₀ alkheterocyclyl, or C₁₋₇ heteroalkyl;
R⁵, R⁶, and R⁷ are each, independently, selected from H, C₁₋₇ alkyl, C₂₋₇ alkenyl, C₂₋₇ alkynyl, C₂₋₆ heterocyclyl, C₆₋₁₂ aryl, C₇₋₁₄ alkaryl, C₃₋₁₀ alkheterocyclyl, or C₁₋₇ heteroalkyl, halide, NO₂, CO₂H, SO₃H, CF₃, CN, OR²⁹, SR³⁰, or are described by the formulas:

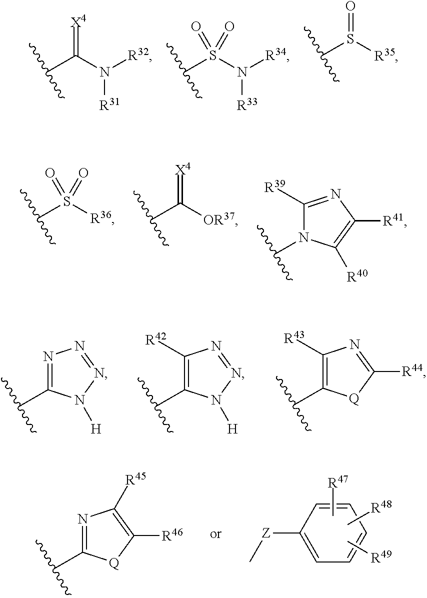

X¹, X², X³, and X⁴ is, independently, O, S; or NR³⁸;
Y is CR²⁵R²⁶, O, S, or NR²⁷;
Z is O, S, or CR⁵⁰R⁵¹;
Q is, independently, O, S, or NR⁵²;
R⁹, R¹⁰, and R¹¹ are each, independently, H, OH, OR¹², C₁₋₇ alkyl, C₂₋₇ alkenyl, C₂₋₇ alkynyl, C₁₋₇ heteroalkyl, halide, or NO₂;
R¹² and R¹³ are each, independently, acyl, C₁₋₇ alkyl, C₂₋₇ alkenyl, C₂₋₇-alkynyl, C₂₋₆ heterocyclyl, C₆₋₁₂ aryl, C₇₋₁₄ alkaryl, C₃₋₁₀ alkheterocyclyl, or C₁₋₇ heteroalkyl;
R¹⁷, R²², R³⁵, R³⁶, R³⁷, R³⁸ and R⁵² are each, independently, C₁₋₇ alkyl, C₂₋₇ alkenyl, C₂₋₇ alkynyl, C₂₋₆ heterocyclyl, C₆₋₁₂ aryl, C₇₋₁₄ alkaryl, C₃₋₁₀ alkheterocyclyl, or C₁₋₇ heteroalkyl;
R¹⁴, R¹⁵, R¹⁶, R¹⁸, R¹⁹, R²⁰, R²¹, R²³, R²⁴, R²⁵, R²⁶, R²⁷, R²⁸, R²⁹, R³⁰, R³¹, R³², R³³, R³⁴, and R⁴⁷ are each, independently, H, C₁₋₇ alkyl, C₂₋₇ alkenyl, C₂₋₇ alkynyl, C₂₋₆ heterocyclyl, C₆₋₁₂ aryl, C₇₋₁₄ alkaryl, C₃₋₁₀ alkheterocyclyl, or C₁₋₇ heteroalkyl; and
R³⁹, R⁴⁰, R⁴¹, R⁴², R⁴³, R⁴⁴, R⁴⁵, R⁴⁶, R⁴⁷, R⁴⁸, R⁴⁹, R⁵⁰, and R⁵¹ are each, independently, H, halide, CN, NO₂, CF₃, C₁₋₇ alkyl, C₂₋₇ alkenyl, C₂₋₇ alkynyl, C₂₋₆ heterocyclyl, C₆₋₁₂ aryl, C₇₋₁₄ alkaryl, C₃₋₁₀ alkheterocyclyl, or C₁₋₇ heteroalkyl, and
   wherein the Wnt/Frizzled-related disease is not a neoplasm.

In an aspect the disclosure provides a method of treating a Wnt/Frizzled-related cardiovascular disease in a subject in need of such treatment, the method comprising administering to the subject an effective amount of a niclosamide compound, or pharmaceutically acceptable salt thereof, of Formula I as described above.

The disclosure provides for and encompasses additional aspects and embodiments, which will be apparent to those of skill in the art in light of the following description.

### BRIEF DESCRIPTION OF THE FIGURES

FIG. 1A is an image of U2OS cells stably expressing Frizzled1-GFP that were treated for 6 hr with control conditioned medium (CTL CM). FIG. 1B is an image of U2OS cells stably expressing Frizzled1-GFP that were treated for 6 hr with Wnt3A conditioned medium (Wnt3A CM). FIG. 1C is an image of U2OS cells stably expressing Frizzled1-GFP that were treated for 6 hr with Wnt5A conditioned medium (Wnt5A CM).

FIG. 2A is an image of Fzd1-GFP stable U2OS cells (Fzd1GFP-U2OS) that were exposed to vehicle (DMSO) for 6 hr at 37° C. FIG. 2B is an image of Fzd1-GFP stable U2OS cells (Fzd1GFP-U2OS) that were exposed to 12.5 μM niclosamide for 6 hr at 37° C. FIG. 2C is the chemical structure of niclosamide. FIG. 2D is an image of vehicle treated Frizzled1-GFP cells. FIG. 2E is an image of cells treated with niclosamide (12.5 μM) obtained from a secondary source (Sigma-Aldrich, St. Louis, Mo.). FIG. 2F is an image of U2OS cells stably expressing Her2-GFP (Her2GFP-U2OS) treated with DMSO. FIG. 2G is an image of U2OS cells stably expressing Her2-GFP (Her2GFP-U2OS) treated with 12.5 μM niclosamide.

FIG. 3 is an immunoblot of Fzd1-GFP stable U2OS cells that were subjected to cell-surface biotin labeling at 4° C. The upper panel shows an immunoblot for biotin-labeled Frizzled1-GFP enriched using a Neutravidin bead pull down and anti-GFP antibody for Frizzled1-GFP detection. The lower panel shows an immunoblot for β-actin. The treatments of surface biotinylation, glutathione, niclosamide, and temperature are indicated above the blots.

FIG. 4A-E are images of Fzd1-GFP stable U2OS cells that were treated with 12.5 μM niclosamide for 0, 1, 2, 4, and 6 hr. FIG. 4F is a graph the number of Frizzled1-GFP containing vesicles in the cytosol at various time points.

FIG. 5A-F are images of Fzd1-GFP stable U2OS cells were treated with vehicle containing DMSO (0 μM) or niclosamide (0.47 μM, 0.94 μM, 1.88 μM, 3.75 μM, and 7.5 μM) for 6 hr. FIG. 5G is a graph of internalized vesicles per cell for varying concentrations of niclosamide.

FIG. 6A is an image of unstimulated U2OS cells containing β₂-adrenergic receptor-RFP (β₂AR-RFP). FIG. 6B is an image of unstimulated U2OS cells containing Fzd1-GFP. FIG. 6C is a merged image of A and B. FIG. 6D-E are images of the receptors in cells stimulated by 0.1 μM isoproterenol (Iso) at 37° C. for 2 hr and 6 hr, with the merged image in FIG. 6F. FIG. 6G-H are images of the receptors in cells stimulated by 12.5 μM niclosamide at 37° C. for 2 hr and 6 hr, with the merged image in FIG. 6I. FIG. 6J-K are images of cells expressing the Fzd1-GFP and exposed to Alexa-546 Transferrin (Tf) at 100 μg/mL and 12.5 μM niclosamide for 2 hr at 37° C., with the merged image shown in FIG. 6L.

FIG. 7A is an immunoblot for endogenous cytosolic Dishevelled-2 in U2OS cells treated with 12.5 μM niclosamide for 6 hr, with molecular weight standards indicated at the right and β-actin serving as a loading control. FIG. 7B is an immunoblot for endogenous cytosolic Dishevelled-2 in U2OS cells treated with niclosamide in a range of 1-7.5 μM.

FIG. 8A is a graph of the induction of Wnt3A stimulated TOPFlash reporter activity with DMSO (vehicle) or 12.5 μM niclosamide (niclo) treatment, wherein CTL CM and Wnt3A CM depict control and Wnt3A conditioned medium, respectively. FIG. 8B is a graph of the induction of Wnt3A stimulated TOPFlash reporter activity with different concentrations of niclosamide. FIG. 8C is an immunoblot for cytosolic β-catenin in U2OS cells treated with 7.5 μM niclosamide, with β-actin serving as a loading control. FIG. 8D is an immunoblot for cytosolic β-catenin in U2OS cells treated with various concentrations of niclosamide, with β-actin serving as a loading control.

FIG. 9 are graphs of cell growth (OD 562 nm) in the presence of niclosamide and oxaliplatin for (A) colorectal cancer cell lines, and (B) colorectal cancer explants, as determined by an MTT assay.

FIG. 10 are graphs of % proliferation of colorectal cancer cells lines in the presence of niclosamide and oxaliplatin over time, as determined by an MTT assay.

FIG. 11 shows the increase of expression of annexin V (+) as a marker of apoptosis in cancer and normal cells with various concentrations of niclosamide, as determined by a flow-based assay after 72 hr of incubation.

FIG. 12 shows the level of β-catenin (clone 7D11), Dvl2 (clone 3F12), and β-actin (clone C-11) levels in HCT116 or CRC57 cells after treatment with various levels of niclosamide.

FIG. 13 are graphs of cell growth (OD 565 nm or OD 562 nm) of HT29 or HCT116 colorectal cancer cell lines in the presence of various levels of niclosamide with or without oxaliplatin, according to MTT assay after 72 hr of incubation.

FIG. 14 is a (A) graph of niclosamide concentration in NOD/SCID mice plasma over time after oral administration, and (B) niclosamide concentration in NOD/SCID mice tumor tissue relative to that in plasma after oral administration.

FIG. 15 shows tumor volume in NOD/SCID mice, which were inoculated with HCT116 or CRC039 colorectal cancer cells, after treatment with various concentrations of niclosamide.

FIG. 16 shows CRC028 or HCT116 colorectal cancer cells in NOD/SCID mice treated with or without niclosamide and stained for Dvl-2 or β-catenin.

### DETAILED DESCRIPTION

In a general sense the disclosure provides for niclosamide compounds and methods comprising the compounds as modulators of Wnt/Frizzled activity and function, and as research tools in the study of the physiological consequences of Wnt signaling, e.g., in cancer, cardiovascular disease, and regeneration at the molecular level. Niclosamide has proven safe in humans when administered for short durations. Niclosamide compounds (including analogs, derivatives, etc.) can provide safe and effective drug therapies for patients with underlying Wnt-directed diseases such as, for example, various cancers and cardiovascular diseases.

As described in the non-limiting and illustrative embodiments below, libraries of FDA-approved drugs were examined for their utility as Frizzled internalization modulators, employing a primary imaged-based GFP-fluorescence assay that used Frizzled1 endocytosis as the readout. As shown in the Examples, it was discovered that the anti-helminthic niclosamide, a drug used for the treatment of tapeworm, promotes Frizzled1 internalization (endocytosis), down regulates Dishevelled-2 protein, and inhibits Wnt3A-stimulated β-catenin stabilization and LEF/TCF reporter activity. Additionally, following niclosamide mediated internalization, the Frizzled1 receptor co-localizes in vesicles containing Transferrin and agonist-activated β₂-adrenergic receptor. Accordingly, niclosamide compounds can serve as negative modulators of Wnt/Frizzled1 signaling by depleting up-stream signaling molecules (i.e. Frizzled and Dishevelled).

As used herein, a “niclosamide compound” includes niclosamide, niclosamide analogs, and niclosamide derivatives, and any combination thereof. Certain non-limiting niclosamide compounds are known and described, e.g., in PCT Publication No. WO 2004/006906 (incorporated herein by reference), and include, but are not limited to, compounds having Formula I:

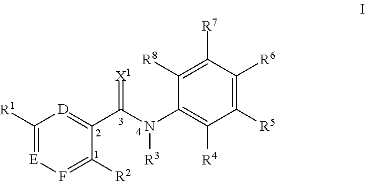

or salts thereof.

In Formula I, D is N or CR⁹; E is N or CR¹⁰; F is N or CR¹¹; and R¹ is H, halide, OR¹², SR¹³NR¹⁴R¹⁵, or described by one of the formulas:

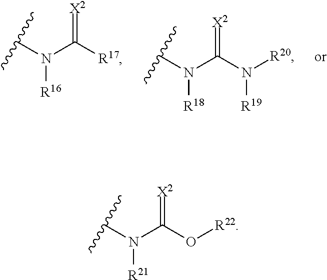

R² is H, OH, or OR¹²;
R³ is H, C₁₋₇ alkyl, C₂₋₇ alkenyl, C₂₋₇ alkynyl, C₂₋₆ heterocyclyl, C₆₋₁₂ aryl, C₇₋₁₄ alkylaryl, C₃₋₁₀ alkylheterocyclyl, or C₁₋₇ heteroalkyl; or
R² and R³ combine to form a six-membered ring in which position 1 is connected to position 4 by one of the groups:

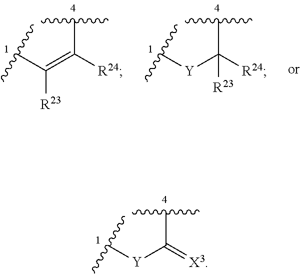

R⁴ and R⁸ are each, independently, selected from H, halide, CF₃, OR²⁸, C₁₋₇ alkyl, C₂₋₇ alkenyl, C₂₋₇ alkynyl, C₂₋₆ heterocyclyl, C₇₋₁₄ alkaryl, C₃₋₁₀ alkheterocyclyl, or C₁₋₇ heteroalkyl;
R⁵, R⁶, and R⁷ are each, independently, selected from H, C₁₋₇ alkyl, C₂₋₇ alkenyl, C₂₋₇ alkynyl, C₂₋₆ heterocyclyl, C₆₋₁₂ aryl, C₇₋₁₄ alkaryl, C₃₋₁₀ alkheterocyclyl, or C₁₋₇ heteroalkyl, halide, NO₂, CO₂H, SO₃H, CF₃, CN, OR²⁹, SR³⁰, or are described by the formulas:

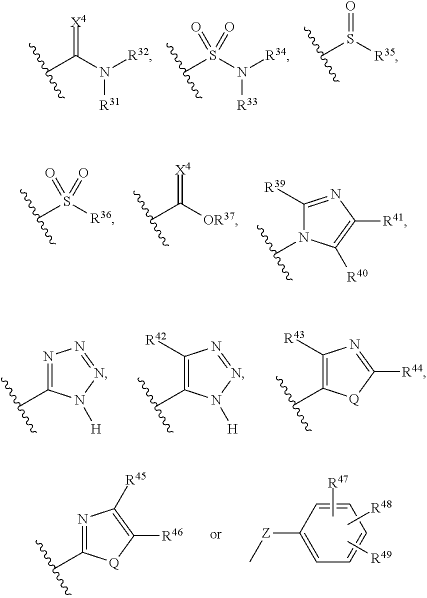

For compounds of Formula I, each X¹, X², X³, and X⁴ is, independently, O, S; or NR³⁸; Y is CR²⁵R²⁶, O, S, or NR²⁷; Z is O, S, or CR⁵⁰R⁵¹; each Q is, independently, O, S, or NR⁵²; R⁹, R¹⁰, and R¹¹ are each, independently, H, OH, OR¹², C₁₋₇ alkyl, C₂₋₇ alkenyl, C₂₋₇ alkynyl, C₁₋₇ heteroalkyl, halide, or NO₂; R¹² and R¹³ are each, independently, acyl, C₁₋₇ alkyl, C₂₋₇ alkenyl, C₂₋₇-alkynyl, C₂₋₆ heterocyclyl, C₆₋₁₂ aryl, C₇₋₁₄ alkaryl, C₃₋₁₀ alkheterocyclyl, or C₁₋₇ heteroalkyl; R¹⁷, R²², R³⁵, R³⁶, R³⁷, R³⁸ and R⁵² are each, independently, C₁₋₇ alkyl, C₂₋₇ alkenyl, C₂₋₇ alkynyl, C₂₋₆ heterocyclyl, C₆₋₁₂ aryl, C₇₋₁₄ alkaryl, C₃₋₁₀ alkheterocyclyl, or C₁₋₇ heteroalkyl; R¹⁴, R¹⁵, R¹⁶, R¹⁸, R¹⁹, R²⁰, R²¹, R²³, R²⁴, R²⁵, R²⁶, R²⁷, R²⁸, R²⁹, R³⁰, R³¹, R³², R³³, R³⁴, and R⁴⁷ are each, independently, H, C₁₋₇ alkyl, C₂₋₇ alkenyl, C₂₋₇alkynyl, C₂₋₆ heterocyclyl, C₆₋₁₂ aryl, C₇₋₁₄ alkaryl, C₃₋₁₀ alkheterocyclyl, or C₁₋₇ heteroalkyl; and R³⁹, R⁴⁰, R⁴¹, R⁴², R⁴³, R⁴⁴, R⁴⁵, R⁴⁶, R⁴⁷, R⁴⁸, R⁴⁹, R⁵⁰, and R⁵¹ are each, independently, H, halide, CN, NO₂, CF₃, C₁₋₇ alkyl, C₂₋₇ alkenyl, C₂₋₇ alkynyl, C₂₋₆ heterocyclyl, C₆₋₁₂ aryl, C₇₋₁₄ alkaryl, C₃₋₁₀ alkheterocyclyl, or C₁₋₇ heteroalkyl.

Certain non-limiting compounds of Formula I include compounds of Formulas II-V:

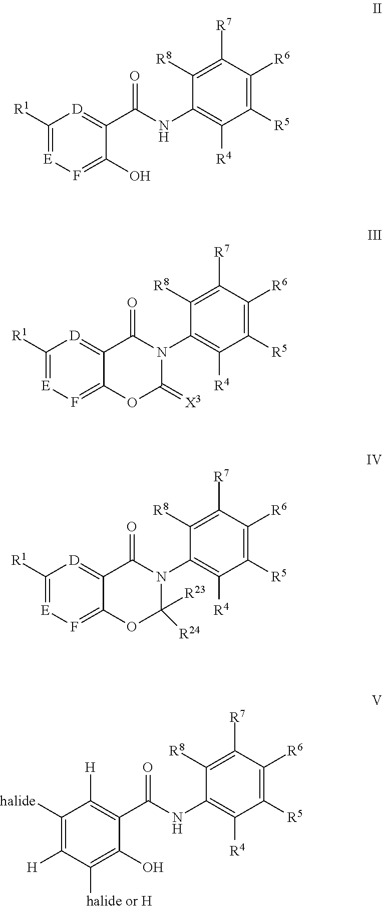

wherein F, E, D, X³, R¹, R⁴, R⁵, R⁶, R⁷, R⁸, R⁹, R¹⁰, R¹¹, R²³, and R²⁴ are as defined above.

In certain embodiments, provided are methods of predicting responsiveness of a Wnt/Frizzled-related disease, such as a neoplasm or cardiovascular disease, to treatment with at least one niclosamide compound, or combinations thereof. The methods may comprise determining the level of at least one protein in the neoplasm, wherein the protein is involved in the Wnt/Frizzled signaling pathway, and comparing the level of the protein to a standard level. An increased level of the protein may indicate that the neoplasm is responsive to treatment with the niclosamide compound. The methods may comprise determining whether the Wnt/Frizzled signaling pathway is dysregulated in diseased tissue and/or cells relative to normal tissue and/or cells. Niclosamide compounds are predicted, in certain embodiments, to be effective for a disease in which the Wnt/Frizzled signaling pathway is dysregulated relative to normal tissue and/or cells.

In some embodiments, the methods can be useful, for example, when a certain type of cancer is actually a group of cancers, each with a different genetic makeup. That is, while two cancers may be identified as the same type, for example, based on histology, they may involve different genetic mutations. In such instances, one cancer may comprise a dysregulated Wnt/Frizzled signaling pathway, while the other cancer does not. The cancer with the dysregulated Wnt/Frizzled signaling pathway would be expected to respond more strongly to treatment with a niclosamide compound than the cancer without the dysregulated Wnt/Frizzled signaling pathway. The cancer that would be expected to respond more strongly to treatment with a niclosamide compound can be identified using the methods described herein.

The level of a protein may be determined by a variety of techniques, as will be appreciated by those of skill in the art. For example, the level of protein expression may be evaluated at either the protein or mRNA level using techniques including, but not limited to, Western blot, ELISA, Northern blot, real time PCR, immunofluorescence, or FACS analysis. The level of a protein may be determined by determining the gene expression level of the protein. For example, the expression level of a protein may be evaluated by immunofluorescence by visualizing cells stained with a fluorescently-labeled protein-specific antibody, Western blot analysis of protein expression, and RT-PCR of protein transcripts

As used herein, “indicative” when used in the context of gene expression levels “that are indicative” means that the gene expression levels are up-regulated or down-regulated, altered, or otherwise changed compared to standard/normal gene expression levels. Similarly, the term “indicative” when used in the context of protein levels means that the protein levels are higher or lower, increased or decreased, altered, or changed compared to standard/normal protein levels.

As used herein, “standard level” refers to the level in a subject, member, or cell of a population that does not have a Wnt/Frizzled-related disease, for example, a subject or member that does not have a cardiovascular disease, cancer, or pre-cancer. The term “standard protein levels” refers to the protein levels in a subject or member of a population that does not have a Wnt/Frizzled-related disease. The factors for determining a population include race, gender, age, geographic location and ethnic origin. In one embodiment, the standard gene expression levels for the genes are the average expression levels of the genes for a non-infected population to which the subject belongs, e.g., adult American female or male, or for a particular subject prior to being infected. A difference between the levels is indicative of a subject having a Wnt/Frizzled-related disease. For example, a peripheral blood sample may be obtained from a subject at a medical laboratory, the blood sample worked up and screened for gene expression, the results of the screening compared to the standards, and the subject informed of her disease status.

Predicting may include using the information found in the Examples or generated by another entity to generate predictions. Predictions may be based on a comparison internal to a single assay or by comparison to a standard. For example the level of expression of a protein may be used to predict a cancer's responsiveness to a therapeutic. Predictions may be generated in relation to a standard or control as discussed above. This does not mean that the predicted event will occur with 100% certainty. Predicting and prediction also includes, but is not limited to, generating a statistically based indication of whether a particular event will occur, e.g. whether the cancer will be responsive to treatment with certain agents.

Proteins involved in the Wnt/Frizzled signaling pathway include, but are not limited to, β-catenin (SEQ ID NO: 2), a Wnt protein, Frizzled (SEQ ID NO: 3), and Dishevelled (Dvl1, SEQ ID NO: 4; Dvl2, SEQ ID NO: 5; Dvl3, SEQ ID NO: 6). Wnt proteins include, but are not limited to, Wnt3A (SEQ ID NO: 1).

A “Wnt/Frizzled-related disease,” as used herein, is a disease in which the Wnt/Frizzled signaling pathway is dysregulated. Certain exemplary Wnt/Frizzled-related diseases include, but are not limited to, cardiovascular disease, neoplasm, obesity, osteoporosis, neuron degeneration, cancer, and disorders in wound healing and tissue repair. The Wnt/Frizzled signaling pathway may be considered dysregulated when, for example, diseased tissue and/or cells comprise at least one of: increased levels of β-catenin; increased LEF/TCF-mediated transcription; increased levels of one or more Wnt proteins, including, but not limited to, Wnt3A; increased levels of Frizzled; and/or increased levels of Dishevelled; as compared to normal tissue and/or cells. As used herein, the term “tissue” includes all biological tissues, including, but not limited to, organ tissue, tumor tissue, skin, blood, etc.

In certain embodiments, a Wnt/Frizzled-related disease is a cardiovascular disease, such as myocardial infarction and cardiac hypertrophy. Cardiovascular disease may further include coronary heart disease (including heart attack and angina pectoris or chest pain); stroke; hypertension, high blood pressure; heart failure; rheumatic fever/rheumatic heart disease; congenital cardiovascular defects; arrhythmias (disorders of heart rhythm); diseases of the arteries, arterioles, and capillaries (including atherosclerosis and Kawasaki disease); bacterial endocarditis; cardiomyopathy; valvular heart disease; diseases of pulmonary circulation; diseases of veins and lymphatics; and other diseases of the circulatory system. In certain embodiments, inhibition of Wnt signaling in such cardiovascular diseases results in a beneficial effect on infarct healing, increased angiogenesis, and/or an attenuated hypertrophic response in the heart.

In certain embodiments, a Wnt/Frizzled-related disease is a neoplasm. In certain embodiments, neoplasm is cancer or a cancer cell. Certain exemplary Wnt/Frizzled-related cancers include, but are not limited to, colon cancer, melanomas, hepatocellular carcinomas, leukemia, ovarian cancer, prostate cancer, lung cancer, brain tumor, and breast cancer.

In certain embodiments, determining whether a cancer comprises a dysregulated Wnt/Frizzled signaling pathway may comprise detecting the level of one or more of Wnt, Frizzled, β-catenin, and/or Dishevelled, and comparing the level to normal tissue and/or cells. In certain such embodiments, if the cancer comprises higher levels of Wnt, Frizzled, β-catenin and/or Dishevelled as compared to normal tissue and/or cells, the cancer is predicted to respond to treatment with a niclosamide compound. In certain embodiments, determining whether a cancer comprises a dysregulated Wnt/Frizzled signaling pathway comprises detecting the level of LEF/TCF-mediated transcription as compared to LEF/TCF-mediated transcription in normal tissue and/or cells. In certain such embodiments, if the cancer comprises a higher level of LEF/TCF-mediated transcription as compared to normal tissue and/or cells, the cancer is predicted to respond to treatment with a niclosamide compound.

In certain embodiments, provided are methods of identifying a subject with a Wnt/Frizzled-related disease. The methods may comprise determining the level of at least one protein in a sample from a subject, wherein the protein is involved in the Wnt/Frizzled signaling pathway, and comparing the level of the protein to a standard level. An increased level of the protein may be indicative of a subject having a Wnt/Frizzled-related disease.

In certain embodiments, provided are methods of treating a subject with a Wnt/Frizzled-related disease. The methods may comprise determining the level of at least one protein in a sample from a subject, wherein the protein is involved in the Wnt/Frizzled signaling pathway, and comparing the level of the protein to a standard level, wherein an increased level of the protein may be indicative of a subject having a Wnt/Frizzled-related disease, and further administering to the subject a niclosamide compound in an amount effective to treat the disease.

“Administration” or “administering” refers to delivery of the compounds by any appropriate route to achieve the desired effect. Administration may include, but is not limited to, oral, sublingual, intramuscular, subcutaneous, intravenous, transdermal, topical, parenteral, buccal, rectal, and via injection, inhalation, and implants.

The term “contacting a cell” is used to mean contacting a cell directly or indirectly in vitro, ex vivo, or in vivo (i.e. within a subject, such as a mammal, including humans, mice, rats, rabbits, cats, and dogs). Contacting a cell, which also includes “reacting” a cell, can occur as a result of administration to a subject. Contacting encompasses administration to a cell, tissue, mammal, subject, patient, or human. Further, contacting a cell includes adding an agent to a cell culture. Other suitable methods may include introducing or administering an agent to a cell, tissue, mammal, subject, or patient using appropriate procedures and routes of administration as defined above.

“Effective amount” refers to a dosage of the compounds or compositions effective for eliciting a desired effect. This term as used herein may also refer to an amount effective at bringing about a desired in vivo effect in an animal, preferably, a human, such as treatment of a disease.

The term “treatment”, as used herein in the context of treating a condition, pertains generally to treatment and therapy, whether of a human or an animal (e.g. in veterinary applications), in which a desired therapeutic effect is achieved. For example, treatment includes prophylaxis and can ameliorate or remedy the condition, disease, or symptom, or treatment can inhibit the progress of the condition or disease (e.g., reduce the rate of disease/symptom progression or halt the rate of disease/symptom progression).

In certain embodiments, provided are methods of detecting a neoplasm in a subject. The methods may comprise determining the level of at least one protein in a sample from a subject, wherein the protein is involved in the Wnt/Frizzled signaling pathway, and comparing the level of the at least one protein to a standard level. An increased level of the protein may be indicative of the subject having a neoplasm.

In certain embodiments, a method of treating cancer comprises first predicting whether or not the cancer will respond to treatment with a niclosamide compound, and if the cancer is predicted to respond, administering a niclosamide compound.

It will be understood that any numerical value recited herein includes all values from the lower value to the upper value. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between the lowest value and the highest value enumerated are to be considered to be expressly stated in this application.

Also, it is to be understood that the phraseology and terminology used herein is for the purpose of description and should not be regarded as limiting. The use herein of terms such as “comprising,” “including,” “having,” and variations thereof is meant to encompass the items listed thereafter and equivalents thereof as well as additional items. “Comprising” encompasses the terms “consisting of” and “consisting essentially of.” The use of “consisting essentially of” means that the composition or method may include additional ingredients and/or steps, but only if the additional ingredients and/or steps do not materially alter the basic and novel characteristics of the claimed composition or method.

All patents publications and references cited herein are hereby fully incorporated by reference.

While the following examples provide further detailed description of certain embodiments of the invention, they should be considered merely illustrative and not in any way limiting the invention, as defined by the claims.

### EXAMPLES

### Example 1

### Materials and Methods

Reagents.

Niclosamide was purchased from Sigma Chemical Co. (St. Louis, Mo.). 7-AAD and Annexin V-biotin kit were purchased from Immunotech (Marseille, France, cat#PN IM3422).

Plasmids, Antibodies, and Conditioned Media.

pCS2ratFrizzled1-GFP (stock #16821) and pLKO.1 (stock #10878) were obtained from Addgene (Cambridge, Mass.). The reporter plasmid p8xTOPFlash was obtained from Dr. Randall Moon. A plasmid for Renilla luciferase was purchased from Promega. β₂-adrenergic receptor-RFP (β₂AR-RFP) was prepared similarly as described for the GFP derivative (Chen, W., et al. Science 2003, 301, 1391-1394; Barak, L. S., et al. Mol Pharmacol 1997, 51, 177-184, both are incorporated herein by reference in their entireties). β-catenin (sc-7963), Dishevelled-1 (sc-8025), Dishevelled-2 (sc-13974, Lot# A242), Dishevelled-3 (sc-8027, and sc-28846), and β-actin (sc-47778) antibodies were obtained from Santa Cruz. The cell lines to produce Wnt3A (CRL-2647), Wnt5A (CRL-2814), and Control (CCL-1.3) conditioned media were obtained from the ATCC. The conditioned media was generated by growing cells in DMEM plus 10% FBS according to the protocol described at http://www.stanford.edu/˜musse/assays/W3aPurif.htm#assay.

Stable Cell Line Generation.

To obtain a Frizzled1-GFP stable cell line (Fzd1GFP-U2OS), U2OS cells were transfected with pCS2ratFrizzled1-GFP and pLKO.1 (10:1 ratio by weight), using the Nucleofection transfection protocol from Amaxa, and stable receptor expressing clones were selected using 1.5 μg/mL puromycin in the culture medium. For generating TOPFlash stable cell lines, HEK293 cells were transfected with p8xTOPFlash, the Renilla luciferase plasmid, and pLKO.1 at a ratio by weight of 10:3:1 and stable clones selected using 1 μg/mL puromycin in the growth medium. pLKO.1 was used to confer puromycin resistance to the stable clones.

Image-Based Primary Screening Assay.

A library containing approximately 1200 FDA approved drugs and drug-like tool compounds was purchased from Prestwick Chemicals Inc. U2OS cells stably expressing Frizzled1-GFP were split into glass-bottom 384 well plates (MGBI01-1-2-LG, MatriCal, Spokane, Wash.) at a density of 6,000 cells/25 μL media/well using a Multidrop 384 dispenser (Titertek Instruments, Huntsville, Ala.). The plates were incubated overnight at 37° C. in 5% CO₂. The following day, chemical compounds (5 mM in DMSO) from the Prestwick library were diluted 1:80 in culture media, 6.25 μL of which were then added to each well of cells using a Biomek FX liquid handler configured with a 96 channel head (Beckman Coulter, San Jose, Calif.) to produce a 1:400 dilution overall and final compound concentration of 12.5 μM per well. The cells were incubated with compound for 6 hr at 37° C. prior to fixation in PBS containing 0.5% paraformaldehyde and 0.002% of the fluorescent nuclear stain DRAQ5. Plates were stored at 4° C. until analysis on an ImageXpress Ultra high throughput imaging system (Molecular Devices, Sunnyvale, Calif.) equipped with a 488 nm argon laser for imaging GFP and a 568 nm krypton laser for imaging DRAQ5. All imaging data were verified by visual inspection and a Z′ factor of 0.44 was calculated for the robustness of the assay.

Cell Surface Biotinylation Internalization Assay.

Frizzled1 internalization was assessed by a surface biotin labeling method (Yang, X. L., et al. Mol Cell Neurosci 2005, 28, 335-346, incorporated herein by reference in its entirety). Fzd1GFP-U2OS cells were grown to confluence in a 6 cm plate, washed twice with PBS containing 10 mM HEPES, incubated at 4° C. for 1 hr with 2 mL of 1 mg/mL sulfo-NHS—S—S-biotin (Pierce, Rockford, Ill.), and washed three times with cold PBS containing 50 μM Tris-HCl. To assess Frizzled1 internalization, the cells were incubated at 37° C. for 4 hr in culture medium with or without 12.5 μM niclosamide, returned to 4° C., and incubated for 15 min twice with fresh glutathione cleavage solution (50 mM reduced L-glutathione, 75 mM NaCl, 10 mM EDTA, 1% BSA, and 0.075 N NaOH) in order to remove biotin remaining on the cell surface. The cells were then washed with cold PBS three times, and lysed with RIPA buffer (50 mM Tris-HCl, PH 8.0, 150 mM NaCl, 1% NP40, 0.5% sodium deoxycholate, 0.1% SDS). Biotinylated proteins from the cell lysate were pulled down with Neutravidin beads (Pierce, Rockford, Ill.), and the beads were eluted with Laemmli SDS loading buffer/50 mM dithiothreitol for 2 hrs at room temperature. Eluted Frizzled1-GFP was identified using SDS-PAGE and anti-GFP antibody. As controls, the surface-biotinylated cells after washing with PBS/50 μM Tris-HCl were either directly lysed with RIPA buffer to assay the total biotin-labeled Frizzled1-GFP, or immediately subjected to glutathione cleavage to monitor the efficiency of the biotin removal.

Image Analysis and Quantification of Internalized Vesicles.

Confocal images were acquired with a Zeiss LSM510 confocal microscope, and analyzed using the computer program Metamorph (Universal Imaging Corporation) as described (Lu, J., et al. Neuron 2007, 55, 874-889, incorporated herein by reference in its entirety). To measure the number of the internalized vesicles per cell, cytosol was carefully traced to exclude cell membrane. Internalized vesicles were defined by setting the threshold of the image to 3-fold of the background intensity. The number of internalized vesicles per cell was counted by the Metamorph software. More than 30 cells per sample were analyzed to obtain statistical significance.

Transferrin Endocytosis Assay.

Cells were serum-starved in MEM for 15 min, and then incubated with Alexa-543 conjugated Transferrin (Tf, 100 μg/mL) together with niclosamide (12.5 μM) for 2 hr at 37° C. To remove remaining surface bound Tf, the cells were exposed to unlabeled transferrin (10 mg/mL) for 2 min at room temperature. The cells were then fixed with 4% paraformaldehye, and imaged with a LSM510 confocal microscope (Zeiss).

TOPFlash Reporter Assay.

For the TOPFlash Luciferase assay the TOPFlash stable cells were seeded in 150 μL growing medium/well in 96-well plates at 100% confluency. Fifty microliters of conditioned medium containing the chemical compounds to be tested or DMSO were added to each well. After an 8 hr treatment, the cells were then washed once with PBS, and lysed with 80 μL MPER solution (Pierce, Rockford, Ill.). Thirty microliters of cell lysate were used for measuring luciferase activity in a 96-well plate reader (FluoStar Optima, BMG Labtech, Chicago, Ill.).

Detection of Cytosolic β-catenin and Dishevelled.

To assay cytosolic β-catenin stabilization and Dishevelled-2 expression, U2OS cells were grown to 100% confluency, and then treated with control conditioned medium or Wnt3A conditioned medium supplemented with DMSO or varying concentrations of niclosamide for 6 hr. After treatment, cytosolic fraction as well as cellular membrane were isolated as described (Mikels, A. J., et al. PLoS Biol 2006, 4, e115, incorporated herein by reference in its entirety). Immunoblots using β-catenin or Dishevelled-2 antibody were used to detect the respective protein levels in cytosol or on membrane, with β-actin immunoblots used for loading controls.

Mice.

NOD.CB17-Prkdc^{scid}/J (NOD/SCID) mice were purchased from Jackson Labs (Bar Harbor, Me.) and bred in the Duke Comprehensive Cancer Center Isolation Facility. All work was performed under a Duke IACUC-approved protocol.

Colorectal Cancer Cell Lines.

Colorectal cancer cell lines, HT29 (ATCC HTB-38), HCT116 (ATCC CCL-247), and CaCO2 (ATCC HTB-37) were purchased from ATCC (Manassas, Va.).

Tumor Cell Isolation from Patients' Colorectal Cancer Specimens and Establishment of Explants in NOD/SCID Mice.

Patients undergoing resection of colorectal cancer metastatic to the liver, which were refractory to standard chemotherapy (including fluorouracil, oxaliplatin and bevacizumab), provided signed informed consent approved by the Duke University Medical Center Institutional Review Board before surgery. After the collection of the colorectal cancer specimen, tissue was minced with a blade into pieces smaller than 2 mm cube and digested overnight with triple enzyme buffer which contains collagenase IV (1 mg/ml, Sigma-Aldrich, St. Louis, Mo.), hyaluronidase (100 μg/mL, Sigma-Aldrich, St. Louis, Mo.) and deoxyribonuclease (20 U/mL, Sigma-Aldrich, St. Louis, Mo.) in RPMI1640 medium. The cells were spun down, washed with PBS three times, resuspended in Hank's Balanced Salt Solution and mixed with Matrigel (BD Biosciences, San Jose, Calif.) at 1:1 ratio. Cells (half of available cells from digestion procedure, typically 1×10⁶ cells) were injected into the back of NOD/SCID mice. After 2-4 month growth in vivo, when tumors reached approximately 1 cm in diameter, the mice were sacrificed and tumors were excised, minced, and put into in vitro culture. Some of the minced cells were injected into the flank of NOD/SCID mice, and serial in vivo passages were performed. Colorectal cancer (CRC) cells growing in vitro were used as target cells of the assays.

MTT Assay.

AsPC-1 tumor cells were cultured with T cells at a 1:5 ratio for 7 days in the presence of MEDI-565 or Cont BiTE (100 ng/mL). On day 7, floating cells were discarded and only adherent AsPC-1 cells were harvested with 0.05% trypsin/EDTA and washed with PBS three times. 1×10⁴ AsPC-1 tumor cells were added to each well of 96 well flat bottom plates in 200 μL of complete RPMI1640 medium. The cells were allowed to adhere to the plates overnight at 37° C. (day 0) and were further incubated for 1, 2, 4 or 7 days. 20 μL of 10×MTT (3-[4,5-dimethylthiazol-2-yl]-2,5-diphenyl-tetrazolium bromide, 5 mg/mL) solution was added to each well, and incubated at 37° C. for 2 hr. The adherent cells were lysed with 150 μL of dimethyl sulfoxide (DMSO) and the optical density (OD) at 550 nm was measured.

Flow-Based Cytotoxicity Assay.

For the cytotoxicity assays, 1×10⁵ tumor cells were put into 12 well flat bottom plates with niclosamide at concentrations ranging from 0.2 μM to 20 μM. After 3 days incubation, all cells were harvested with 0.05% trypsin/EDTA and spun down by centrifugation. Cells were labeled with biotin-conjugated Annexin V, and then stained with 7-AAD and Streptavidin-APC. Samples were acquired by FACSCalibur machine (BD Bioscience) and analyzed with CellQuest software. Cells were analyzed for expression of Annexin V as a marker of apoptosis.

Western Blot Analysis.

CRC explants and colorectal cancer cell lines were cultured in 6-well plates. When cells are subconfluent in the wells, they were treated with different concentrations (0, 0.4, 1, 2, 5, 10 μM) of niclosamide overnight (18 hr). After washing cells with PBS three times, hypotonic lysis buffer consist of 10 mM Tris-HCl, pH7.4 and 0.2 mM MgCl₂, supplemented with 1× Halt Protease and Phosphatase Inhibitor Cocktail (Thermo Scientific Pierce Protein Research Products, Rockford, Ill.) were applied to make cell lyates for Western Blot.

Immunohistochemistry.

Tumor grown in the flank of NOD/SCID mice treated with/without niclosamide were excised and fixed with 10% neutralized buffered formalin. Paraffin-embedded specimens were cut into 4 μm-thick of sequential sections. After dewaxing in xylene and rehydrating stepwise in ethanol, sections were subjected to heat-induced antigen retrieval. Endogenous peroxidase activity was blocked with 3% H₂O₂, and non-specific binding was blocked with non-immune horse sera. Sections were then incubated with primary antibodies overnight at 4C. Anti-β-catenin (sc-65483, Santa Cruz Biotechnology, Inc., Santa Cruz, Calif.) and anti-Dvl-2 (sc-8026, Santa Cruz Biotechnology, Inc., Santa Cruz, Calif.) monoclonal antibodies were used at (1:50) dilution. After rinsing the sections were incubated with the biotinylated secondary antibody (1:200 dilution; Dakopatts, Copenhagen, Denmark) for 30 min at room temperature and then with the avidin-biotin peroxidase complex from the Vector Elite ABC kit (Vector Laboratories, Burlingame, Calif.) according to the manufacturer's instructions. Visualization was achieved using 3,3′-diaminobenzidine tetrahydrochloride and H₂O₂. Specimens were counterstained with hematoxylin.

Pharmacokinetic Analysis of Niclosamide.

NOD/SCID mice (weighting 23 to 25 g) received oral administration of niclosamide (100 mg/kg of body weight). Blood samples were obtained from vena cava following euthanasia by ketamin injection at predose and at 0.25, 0.5, 0.75, 1, 1.5, 4, 8, 12, 24 hr after drug administration. Blood samples were put into eppendorf tubes containing 10 μL of heparin (1000 U/mL), centrifuged at 1,200 g for 10 min. Plasma were collected and frozen at −20° C. until HPLC analysis. Concentrations of niclosamide in mouse plasma were determined by an HPLC method.

HPLC.

Quantification of niclosamide in mouse plasma and tumor tissue was guided by the published LC/MS/MS method from Yi-Wei Chang et al. (J. Food Drug Anal, 2006, 14:4, 329-333, incorporated herein by reference in its entirety) which was modified to increase detection limit in plasma, enable measurement in tumor tissue, and adopt the assay to available equipment. A Shimadzu 20A series LC system and Applied Biosystems API 4000 QTrap tandem mass spectrometer were used with the following parameters: Column: Agilent Eclipse 50×4.6 mm, 1.8 mm; mobile phase A: 10 mM ammonium acetate, 0.1% formic acid in water; mobile phase B: methanol; flow: 1 mL/min; column temperature: 50° C.; injection: 5 mL; elution gradient: 0-2 min 50-90% B, 2-4.5 min 90% B, inject 100 mL methanol (carryover wash), 4.5-8.5 min 90% B, 8.5-9 min 90-50% B, 9-13 min 50% B; MS/MS transitions: 327/289 for niclosamide and 229/185 for naproxen (internal standard). The Calibration curve was linear from 0.2 ng/mL (lower limit of quantitation, LLOQ) to 300 ng/mL.

Niclosamide Concentrations in Tumor Tissue.

NOD/SCID mice were inoculated with HCT116 tumor cells (5×10⁶ cells), and on day 4, oral administration (gavage) of niclosamide (200 mg/kg body weight) or control solvent was initiated. After 3-week treatment, mice were sacrificed 24 hr after the last oral administration, and blood and tumor tissue were collected for the analysis of niclosamide concentration. Plasma was isolated from blood as described above. Tumor tissue was frozen in liquid nitrogen, smashed into small pieces/powders, and homogenated with three volumes of deionized water in 2 mL polypropylene tubesa in the FastPrep apparatus (4 mm ceramic bead, 20 s, speed 4; Qbiogene, Montreal, Canada); 50 μL aliquots were stored at −80° C. until analysis. Concentrations of niclosamide in tissue were also determined by HPLC analysis.

In Vivo Anti-tumor Effect of Niclosamide.

HCT116 colon cancer cells were harvested from flasks with 0.05% Trypsin/EDTA, washed with PBS, and resuspended with Hanks buffered solution at 5×10⁶ cells/100 μL concentration. CRC explants (CRC039) cultured in vitro were harvested with the same procedure, and mixed with equal volume of Matrigel to make 1×10⁶ cells/100 μL concentration. A hundred μL of cell suspension was inoculated to the flank of NOD/SCID mice 4 days before the start of treatment. Oral administration of niclosamide was performed with gavage technique, for 6 days/week for 2 (HCT116) or 3 weeks (CRC039). Tumor size was measured 3 times a week until mice were sacrificed.

### Example 2

### Generation of a U2OS Cell Line Stably Expressing Frizzled1-GFP

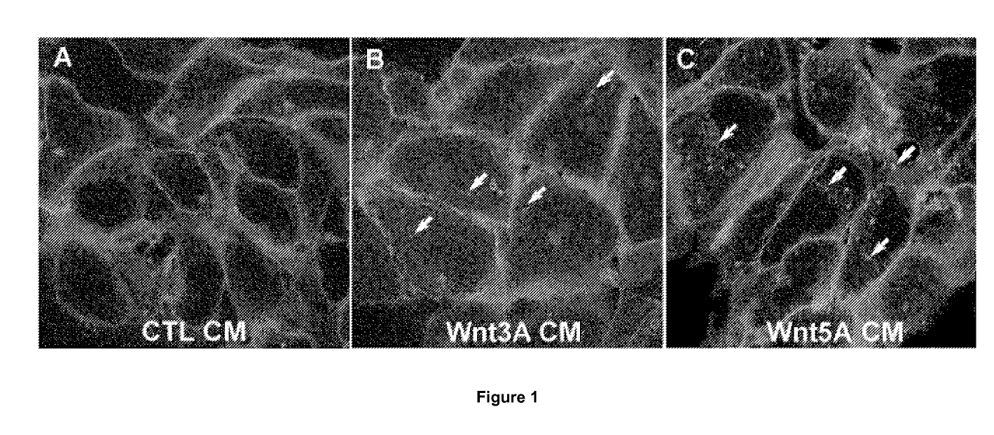

In order to screen small molecule modulators of Frizzled receptor internalization and develop an assay compatible with high throughput screening, a U2OS cell line was generated stably that expresses Frizzled1-GFP (Fzd1GFP-U2OS). U2OS cells stably expressing Frizzled1-GFP were treated for 6 hr with control conditioned medium (CTL CM; FIG. 1A), Wnt3A conditioned medium (Wnt3A CM, FIG. 1B), and Wnt5A conditioned medium (Wnt5A CM; FIG. 1C). The cellular distributions of Frizzled1-GFP chimeras were initially assessed by confocal microscopy, and cells were imaged using a LSM510 confocal microscope (Zeiss) with a IOOX objective and 488 nm excitation. In FIG. 1, internalized vesicles are indicated by arrowheads. Frizzled1-GFP localized predominantly to the plasma membrane with almost no internalized vesicles present when the cells were not stimulated with Wnt ligands (FIG. 1A). When treated with Wnt3A conditioned medium, the cells showed a minimal internalization of receptor fluorescence (FIG. 1B), whereas cells exposed to Wnt5A conditioned medium demonstrated a moderate amount of intracellular fluorescence (FIG. 1C). These observations indicated that Frizzled1 internalization could provide a readout for agonist/ligand activity.

### Example 3

### Screen of FDA-Approved Compounds in Frizzled Internalization Assay

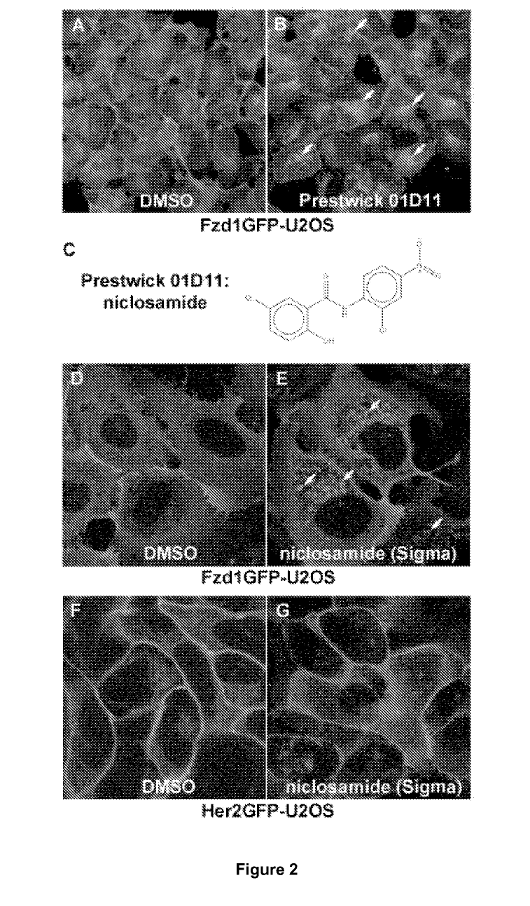

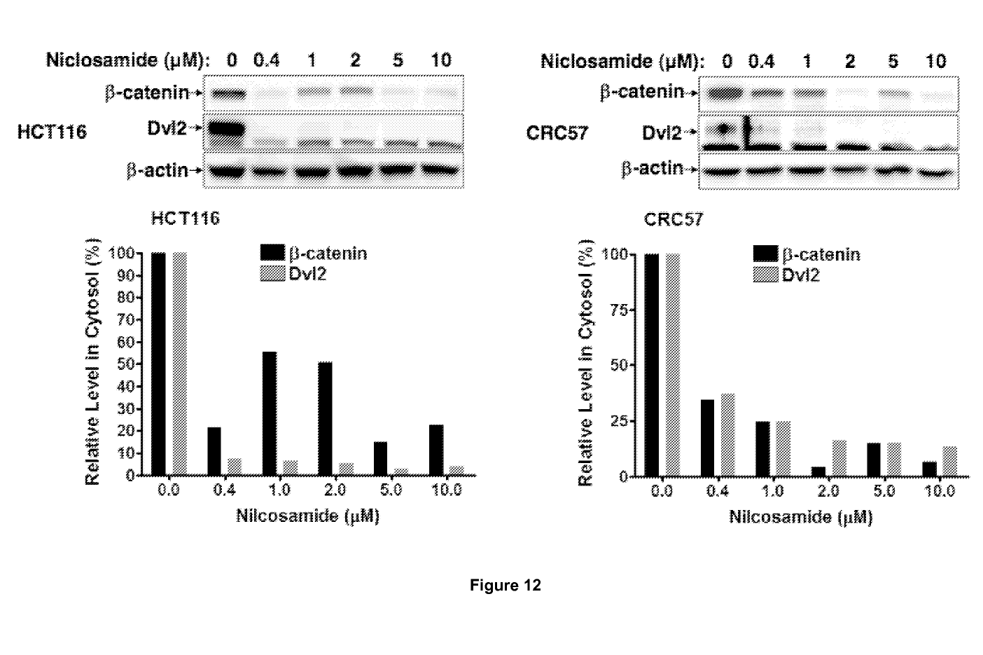

Fzd1-GFP stable U2OS cells (Fzd1GFP-U2OS) were exposed to over 1200 FDA-approved drug and drug-like compounds from the Prestwick Chemical Library at a concentration of 12.5 μM for 6 hr at 37° C. in a 384-well format. Cells were imaged with an ImageXpress Ultra high throughput confocal imaging system using a 40× objective and 488 nm excitation. FIG. 2A shows vehicle (DMSO) treated cells, FIG. 2B shows cells exposed to niclosamide (Prestwick O1D11, 12.5 μM), and internalized vesicles are indicated by arrowheads. FIG. 2C shows the chemical structure of niclosamide. This primary screen revealed that niclosamide (Prestwick O1D11) stimulated internalization of Frizzled1-GFP, and that niclosamide produced much more robust internalization (FIG. 2A-2C) than even Wnt3A or Wnt5A stimulation (FIGS. 1B and 1C). To verify the result, the Fzd1GFP-U2OS cells were also treated with niclosamide obtained from an alternate supplier (Sigma-Aldrich, St. Louis, Mo.), and a similarly strong internalization of Frizzled1-GFP was observed. FIG. 2D shows Vehicle treated Frizzled1-GFP cells, FIG. 2E shows cells treated with niclosamide (12.5 μM) obtained from a secondary source (Sigma-Aldrich, St. Louis, Mo.), and vesicles are indicated by arrowheads. Cells were imaged with a Zeiss LSM510 confocal microscope using a 100× objective and 488 nm excitation. As a control, U2OS cells stably expressing on the plasma membrane the human EGF Receptor 2-GFP (Her2-GFP) were treated with DMSO (FIG. 2F) or 12.5 μM niclosamide (FIG. 2G). Niclosamide did not induce the Her2-GFP-U2OS cells to internalize Frizzled1-GFP.

During the primary screening, an additional 25 small molecule compounds were identified (Table 1) that had some effect on Frizzled1-GFP internalization, but little or no effect on Wnt signaling was observed, as assessed by the TOPFlash luciferase reporter assay (data not shown). For the data in Table 1, the internalization of Frizzled1 was scored visually, and the names and molecular weights of the chemical compound hits are listed. These 25 compounds were therefore not studied further, and niclosamide, which demonstrated a potential for modulating Wnt signaling, was investigated in detail further.

TABLE 1
Summary of Frizzledl1-GFP Internalization Screening

| Internalization Score | |
|---|---|
| (+ to +++, | |
| low to high) | |
| (Number of hits) | Compound Hit name (Molecular Weight, dalton) |
| +++ | Niclosamide (327.12571); |
| (4 hits) | Quinacrine dihydrochloride dehydrate (508.92067); |
| | Lasalocid sodium salt (612.78651); |
| | Tetrandrine (622.76824) |
| ++ | Perhexiline maleate (393.57158); |
| (3 hits) | Fendiline hydrochloride (351.92337); |
| | Amiodarone hydrochloride (681.78455); |
| + | Triflupromazine hydrochloride (388.8857); |
| (19 hits) | Cyproheptadine hydrochloride (323.86919); |
| | Alverine citrate salt (473.57135); |
| | Chlorpromazine hydrochloride (355.33235); |
| | Perphenazine (403.97787); |
| | Dicyclomine hydrochloride (345.95727); |
| | Clomipramine hydrochloride (351.32253); |
| | Amodiaquin dihydrochloride dehydrate (464.82346): |
| | Metixene hydrochloride (363.95332); |
| | Hycanthone (356.49048); |
| | Acetopromazine maleate salt (442.53807); |
| | Clomiphene citrate (Z,E) (598.09862); |
| | Bepridil hydrochloride (403.01235); |
| | Flupentixol digydrochloride cis-(Z) (507.44964); |
| | Prenylamine lactate (417.59665); |
| | Nitrarine dihydrochloride (380.36429); |
| | Monensin sodium salt (692.87077); |
| | Zuclopenthixol hydrochloride (437.43532); |
| | Thiethylperazine malate (667.80337) |

### Example 4

### Internalization of Biotinylated Frizzled1-GFP in the Presence of Niclosamide

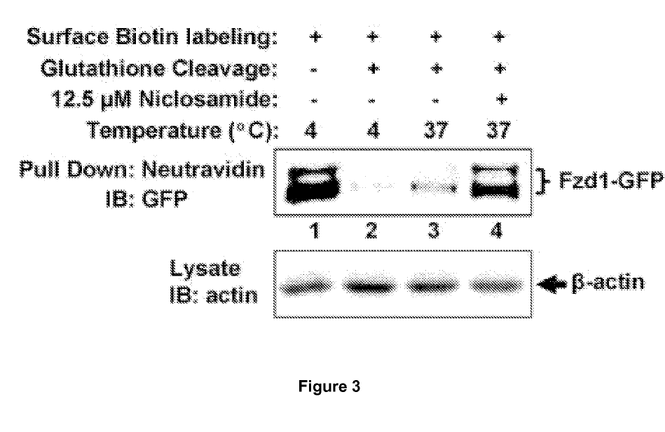

To assess the effect of niclosamide on Frizzled1 internalization, a method was employed independent of and complementary to the primary screening methodology using biotin labeling of the Frizzled1-GFP plasma membrane receptors. First, Fzd1GFP-U2OS cells were surface biotinylated at 4° C. to label only the cell surface receptor population. Next the labeled cells were incubated at 37° C. to allow receptor internalization in the presence of niclosamide. Receptors that internalized in this assay would have their biotin label protected from glutathione cleavage and can be visualized using anti-GFP immunoblots. The upper panel of FIG. 3 shows an immunoblot (IB) for biotin-labeled Frizzled1-GFP enriched using a Neutravidin bead pull down and anti-GFP antibody for Frizzled1-GFP detection. The lower panel of FIG. 3 shows an immunoblot (IB) for β-actin and served as a loading control to ensure that an equal amount of cell lysate was used for the Neutravidin pull down. The treatments of surface biotinylation, glutathione, niclosamide, and temperature are indicated above the blots in FIG. 3. In FIG. 3, Lane 1 shows the total biotin labeled cell surface Frizzled1-GFP, and the nearly complete removal of Frizzled1 bands in Lane 2 demonstrated that receptors on the cell surface (4° C., not internalized) were susceptible to glutathione cleavage of their biotin label. In contrast, biotinylated Frizzled1-GFP receptors exposed to niclosamide at the permissible internalization temperature of 37° C. (FIG. 3, Lane 4) produces a strong immunoblot signal compared to cells not treated with niclosamide (FIG. 3, Lane 3), indicating that the niclosamide-internalized Frizzled1 receptors originated on the plasma membrane.

### Example 5

### Time-Course of Frizzled1-GFP Internalization in the Presence of Niclosamide

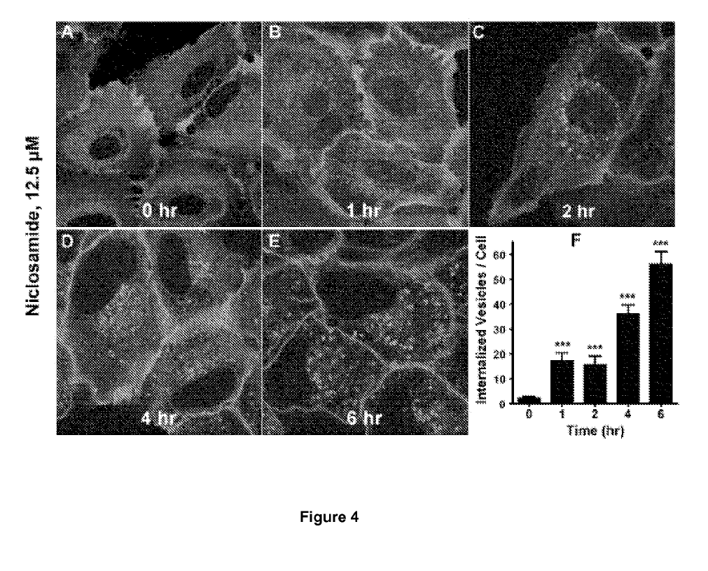

Fzd1-GFP stable U2OS cells were treated with niclosamide (12.5 μM) for 6 hr with samples taken at 0, 1, 2, 4, and 6 hr time points. To determine the time-course of internalization for Frizzled1-GFP receptors, the accumulation of cytosolic puncta/vesicles was measured (FIG. 4A-4E). Internalization was assessed by counting the number of Frizzled1-GFP containing vesicles in the cytosol using the computer program Metamorph, and the results are presented in the graph for the given times (0 hr, n=51 cells; 1 hr, n=69; 2 hr, n=32; 4 hr, n=55, and 6 hr, n=61). Paired data against time 0 hr were analyzed by Students t-test with a significance of P≦0.0005. FIG. 4F is a graphical representation of the internalization time course showing a t_{1/2} of 2.4±0.5 hr for the niclosamide-induced Frizzled1 internalization.

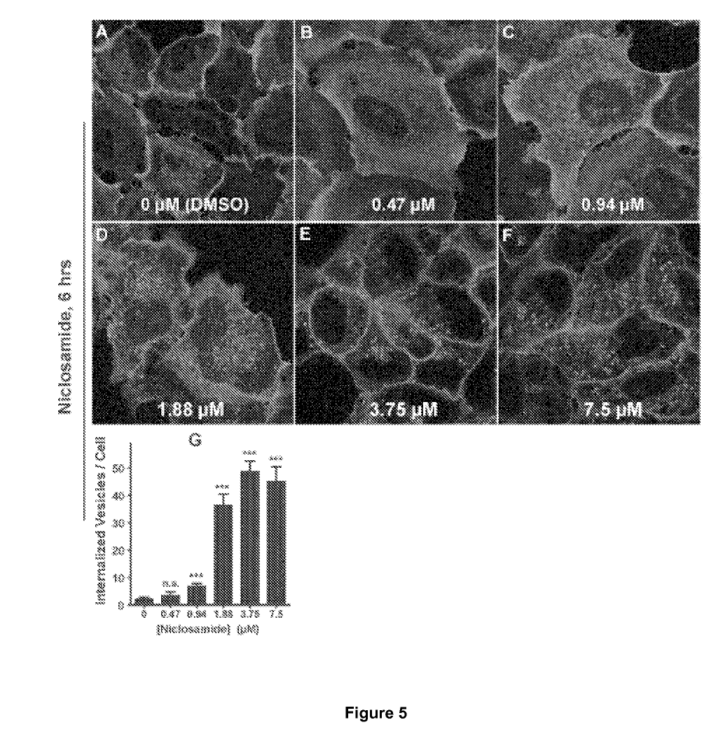

Fzd1-GFP stable U2OS cells were also treated with vehicle containing DMSO (0 μM) or varying concentrations of niclosamide (0.47 μM, 0.94 μM, 1.88 μM, 3.75 μM, and 7.5 μM) for 6 hr. Vesicles per cell were quantified as described for FIG. 4 for the respective number of cells corresponding to an individual dose: n=51, 42, 75, 64, 55, and 57. Data were analyzed paired against 0 μM sample using Students t-test with a significance of P≦0.0005. FIG. 5A-5G shows the dose-dependence at the 6 hr time point of Frizzled1-GFP internalization in the presence of increasing concentrations of niclosamide. At 1-2 μM niclosamide concentration, a significant increase in the number of internalized receptors was observed, which indicated that the potency for niclosamide-induced internalization is in the low micromolar range (FIG. 5G).

### Example 6

### Frizzled1-GFP co-localization with β-adrenergic Receptor-RFP in the Presence of Niclosamide

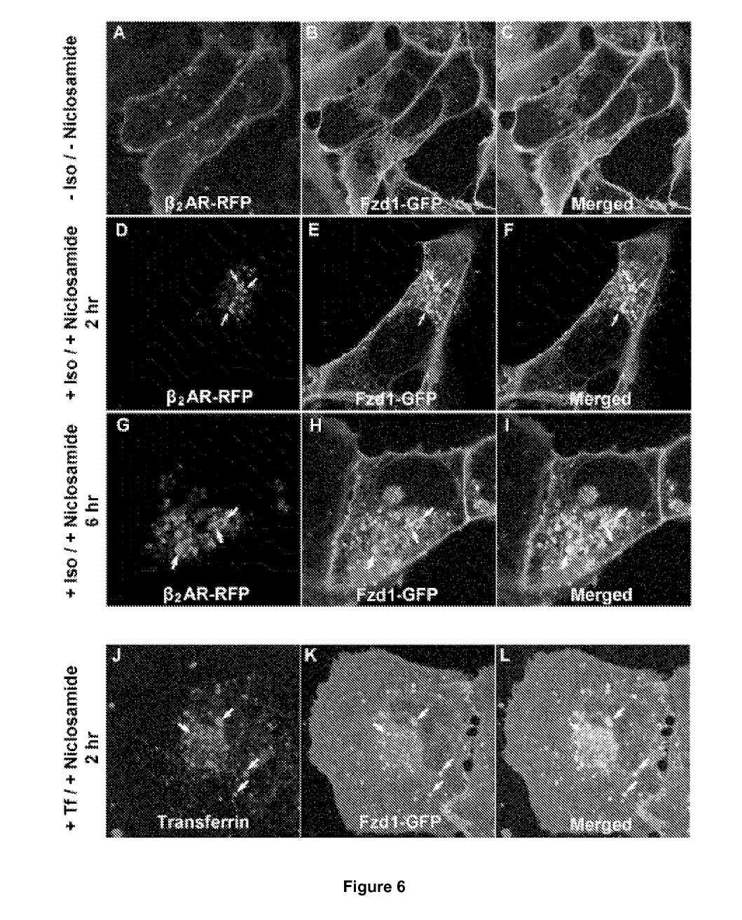

Many classes of membrane receptors internalize in clathrin coated pits. In particular, β₂-adrenergic receptors are prototypical for clathrin-dependent internalization of G protein-coupled receptors (Barak, L. S., et al. Mol Pharmacol 1997, 51, 177-184; Goodman, O. B., Jr., et al. Nature 1996, 383, 447-450, both are incorporated herein by reference in their entireties), and Transferrin is a well-documented standard for clathrin-mediated internalization in general (Mellman, I. Annu Rev Cell Dev Biol 1996, 12, 575-625, incorporated herein by reference). It was examined whether internalized Frizzled1-GFP co-localizes with either β₂-adrenergic receptor-RFP or Transferrin in intracellular vesicles. FIG. 6A-6C show confocal images of unstimulated U2OS cells expressing both β₂-adrenergic receptor-RFP (β₂AR-RFP, FIG. 6A) and Frizzled1 receptors (Fzd1-GFP, FIG. 6B) prior to activation. FIG. 6C shows the merged image, indicating that under these conditions the two receptors were not intracellularly co-localized.

Cells were exposed to 0.1 μM isoproterenol (Iso, FIG. 6D-F) and 12.5 μM niclosamide (FIG. 6G-I) at 37° C. for 2 hr and 6 hr. The merged images are presented in FIG. 6F and FIG. 6I. Exposure to isoproterenol and niclosamide for 2 hr or 6 hr (FIG. 6D-6I) resulted in multiple overlapping intracellular distributions of each receptor.

A similar study was conducted in cells expressing the Fzd1-GFP receptor and exposed to Alexa-546 Transferrin (Tf) at 100 μg/mL and 12.5 μM niclosamide for 2 hr at 37° C. The merged image is presented in (FIG. 6L), wherein arrowheads indicate co-localized vesicles. Internalized Transferrin at 2 hr showed significant co-localization with internalized Frizzled1 (FIG. 6J-6L). These data suggested that niclosamide-induced Frizzled1 internalization occurred through clathrin-coated pits.

### Example 7

### Niclosamide Inhibits the Cytosolic Expression of Endogenous Dishevelled-2

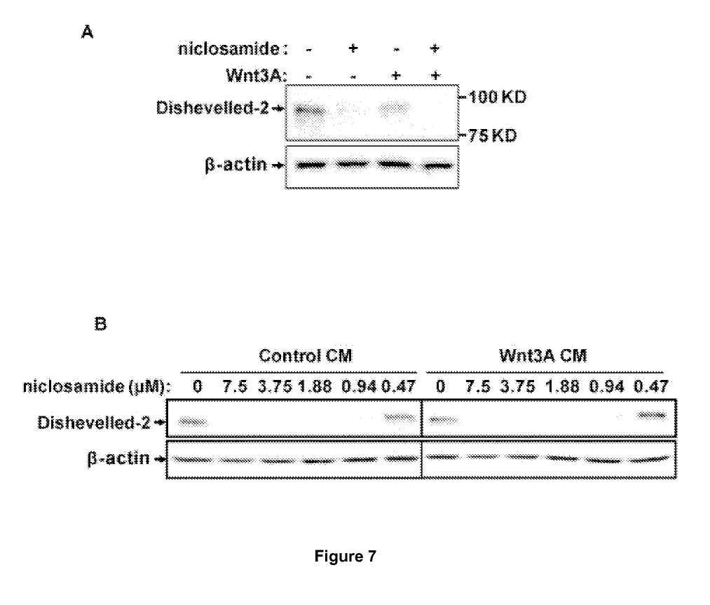

Dishevelled proteins (Dishevelled-1, 2, and 3 in mammalian cells) are intracellular molecules transducing Frizzled signaling. To assess the effect of niclosamide on Wnt signaling mechanism, protein expression of Dishevelled was examined in U2OS cells treated with 12.5 μM niclosamide for 6 hr. In U2OS cells stimulated with either control or Wnt3A conditioned medium, treatment of niclosamide for 6 hr resulted in dramatic reduction of the level of endogenous cytosolic Dishevelled-2 protein, as demonstrated by immunoblotting (FIG. 7A, wherein molecular weight standards are indicated at the right, with β-actin serving as a loading control). The half maximal reduction of Dishevelled-2 occurred at a niclosamide concentration of approximately 1 μM, as shown by the immunoblot of FIG. 7B. No endogenous Dishevelled-2 was detected in the membrane fraction, and endogenous Dishevelled-1 and 3 were not detectable using commercial antibodies.

### Example 8

### LEF/TCF Transcription Factor Activity in the Presence of Niclosamide

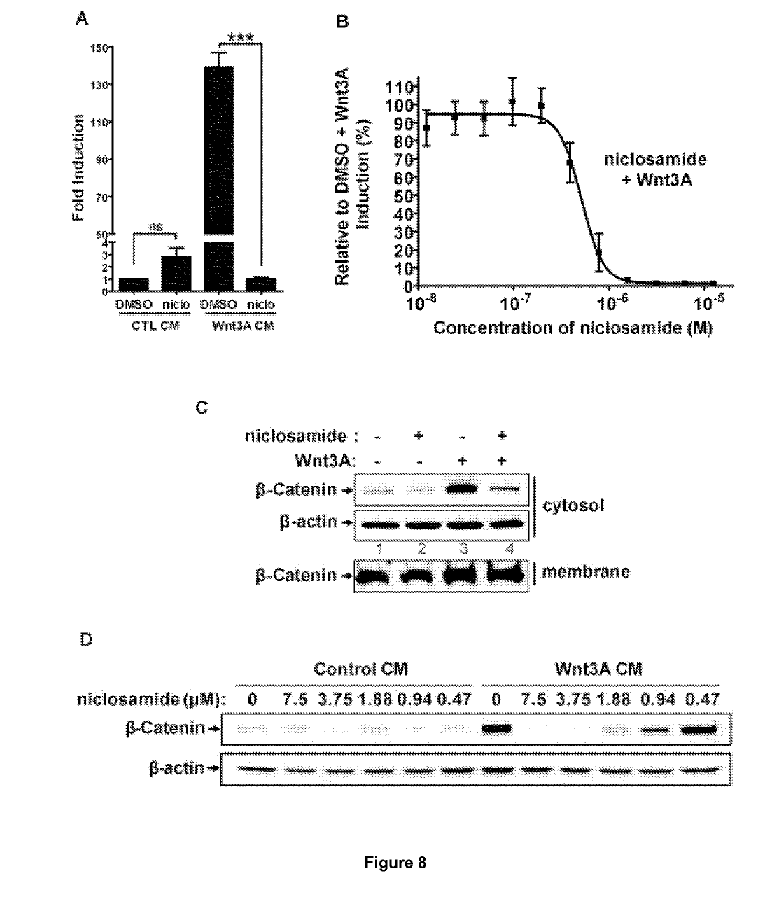

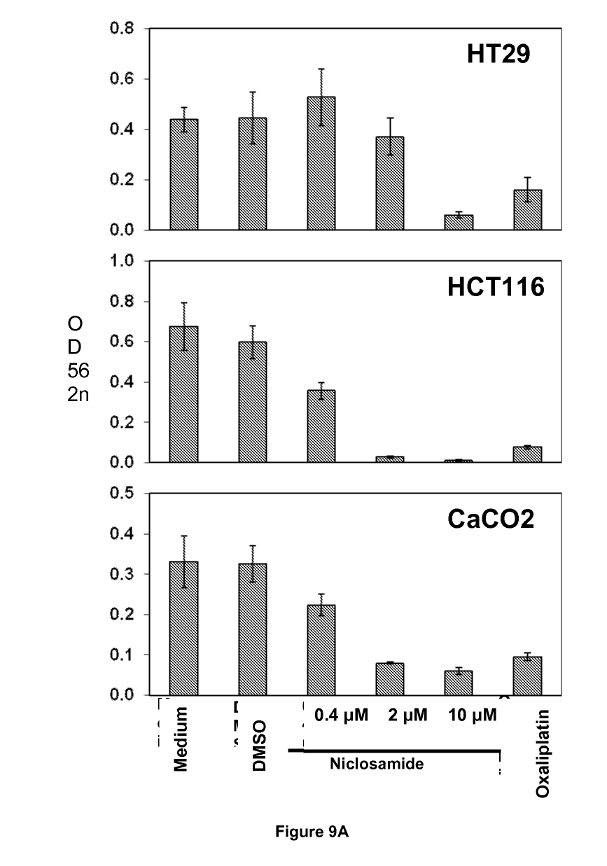

LEF/TCF transcription factor reporter (TOPFlash) assay is a general readout for canonical Wnt signaling pathways. To assess the effect of niclosamide on Wnt signaling activity, an HEK293 cell line was generated that stably expressed a LEF/TCF transcription factor reporter plasmid (TOPFlash) that responds to Wnt mediated β-catenin induction. A Renila luciferase plasmid was also generated to serve as an internal control. HEK293 cells stably expressing TOPFlash luciferase reporter and Renilla luciferase were treated with control conditioned medium or Wnt3A conditioned medium in the presence of DMSO (vehicle) or niclosamide (niclo). The inhibitory effect of 12.5 μM niclosamide on Wnt3A stimulated TOPFlash reporter activity is shown in FIG. 8A (ns, not significant (P>0.05); ***, P<0.0001, t-tests), wherein CTL CM and Wnt3A CM depict control and Wnt3A conditioned medium, respectively. The ability of niclosamide to promote Frizzled1 internalization suggested agonist or partial agonist-like behavior. Niclosamide alone does not produce a statistically significant increase in the TOPFlash (LEF/TCF) reporter signal (FIG. 8A). Upon Wnt3A stimulation, a 140 fold induction of the LEF/TCF reporter signal was observed (FIG. 8A). Remarkably, the addition of niclosamide to the Wnt3A conditioned medium blocked the increase of the reporter signal observed with Wnt3A alone (FIG. 8A), indicating niclosamide inhibits Wnt/Frizzled signaling induced by a full agonist (Wnt in this case).

The study was repeated with varying concentrations of niclosamide. The average TOPFlash reporter activity of Wnt3A plus DMSO treatment was set as 100%, and the relative reporter activity of Wnt3A plus the indicated concentration of niclosmide was calculated and plotted. As shown in FIG. 8B, the inhibitory effect was dose-dependent with an IC₅₀ of 0.5±0.05 μM.

### Example 9

### Cytosolic β-catenin Levels in Presence of Niclosamide

The accumulation of cytosolic β-catenin is a measure of canonical Wnt signaling (Mikels, A. J., and Nusse, R. PLoS Biol 2006, 4, e115, incorporated herein by reference). The reporter assay indicated that Wnt inducible cytosolic β-catenin accumulation should be reduced in the presence of niclosamide. FIG. 8C demonstrates that niclosamide prevented Wnt3A-stimulated cytosolic β-catenin stabilization in U2OS cells as demonstrated by immunoblotting of cytosolic β-catenin (compare Lane 3 and 4, upper panel, cytosol). β-actin was used as a loading control. However, the membrane-bound β-catenin levels were relatively unchanged (FIG. 8C, bottom panel, membrane), indicating the reduction in the signaling β-catenin pool was not due to a loss of β-catenin expression. The potency of niclosamide inhibition in Wnt-mediated β-catenin stabilization was determined from the dose dependence presented in the immunoblots of FIG. 8D. FIG. 8C shows the effect of 7.5 μM niclosamide treatment, whereas FIG. 8D shows that the inhibitory effect occurred over a range of 1-7.5 μM niclosamide (n=3). The half maximal inhibition of Wnt3A signaling occurred at a niclosamide concentration of approximately 1 μM.

In summary, the data indicated that niclosamide promoted Frizzled1 internalization, down regulated the expression of Dishevelled-2 protein, and inhibited Wnt3A-stimulated LEF/TCF (TOPflash) reporter activity and β-catenin stabilization. Therefore niclosamide functions as an inhibitor for Wnt signaling.

### Example 10

### Cytotoxic Effect of Niclosamide on Colorectal Cancer Explants and Cell Lines

Three colorectal cancer cell lines were used in this study: HT29, CaCO₂, and HCT116. HT29 and CaCO2 express mutant APC and also β-catenin without mutation. HCT116 cells express mutant β-catenin and also APC without mutation. All three express Frizzled-1 and Frizzled-2.

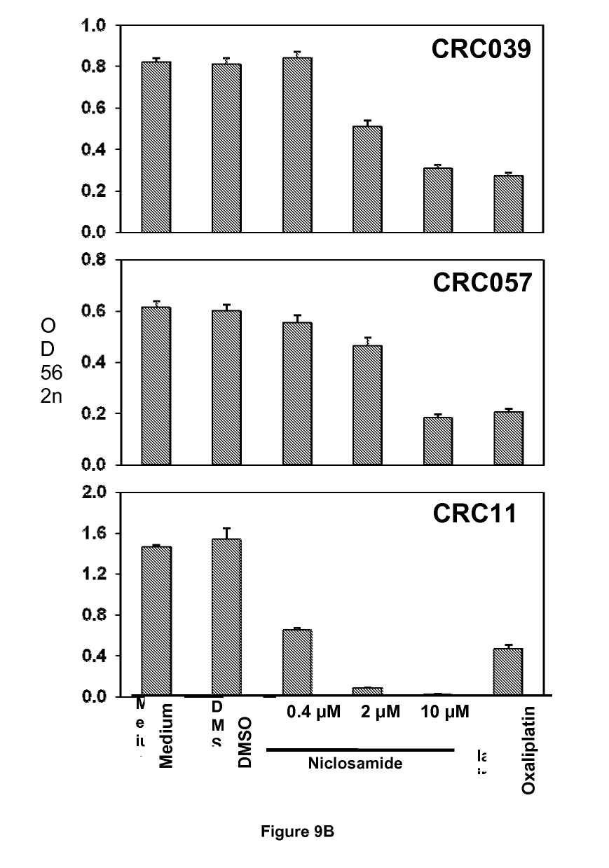

Colorectal cancer cell lines were incubated in 96-well flat-bottomed plates at 5,000 cells per well in 200 μL of complete RPMI1640 medium. The cells were allowed to adhere to the plates overnight at 37° C. (day 0). Several doses of niclosamide (0.4 μM, 2 μM, 10 μM) or 10 μM of oxaliplatin (as a control) were added, and the cells were further incubated for 3 days (72 hr). Then 20 μL of 10×MTT solution was added to each well and incubated at 37° C. for 2 hr. The adherent cells were lysed with 150 μL of DMSO, and then optical density (OD) at 562 nm was measured. DMSO or culture medium was used as a control. All three cell lines showed less proliferation in the presence of niclosamide (FIG. 9A). HCT116 cells were most sensitive, while HT29 cells were less sensitive to niclosamide, only showing anti-proliferation effect at the highest concentration (10 μM). Niclosamide, even at 2 μM concentration, showed stronger cytotoxic effect for HCT116 and CaCO2 cells than 10 μM of oxaliplatin.

The cytotoxic effects of niclosamide against primary cultured cells were analyzed with 8 different CRC explants (CRC007, CRC010, CRC020, CRC025, CRC028, CRC039, CRC057, CRC119). CRC explants were incubated in 96-well flat-bottomed plates at 10,000 cells per well in 200 μL of complete RPMI1640 medium. The cells were allowed to adhere to the plates overnight at 37° C. (day 0). Several doses of niclosamide (0.4 μM, 2 μM, 10 μM) or 10 μM of oxaliplatin (as a control) were added, and the cells were further incubated for 3 days (72 hr). Then 20 μL of 10×MTT solution was added to each well and incubated at 37° C. for 2 hr. The adherent cells were lysed with 150 μL of DMSO, and then optical density (OD) at 562 nm was measured. DMSO or culture medium was used as a control. All CRC explants showed cytotoxic effects of niclosamide, and representative cases are shown in FIG. 9B. A dose-dependent effect of niclosamide was observed for all CRC explants, with some variation of sensitivity among them.

### Example 11

### Over Time Effect of Niclosamide on Colon Cancer Cell Lines

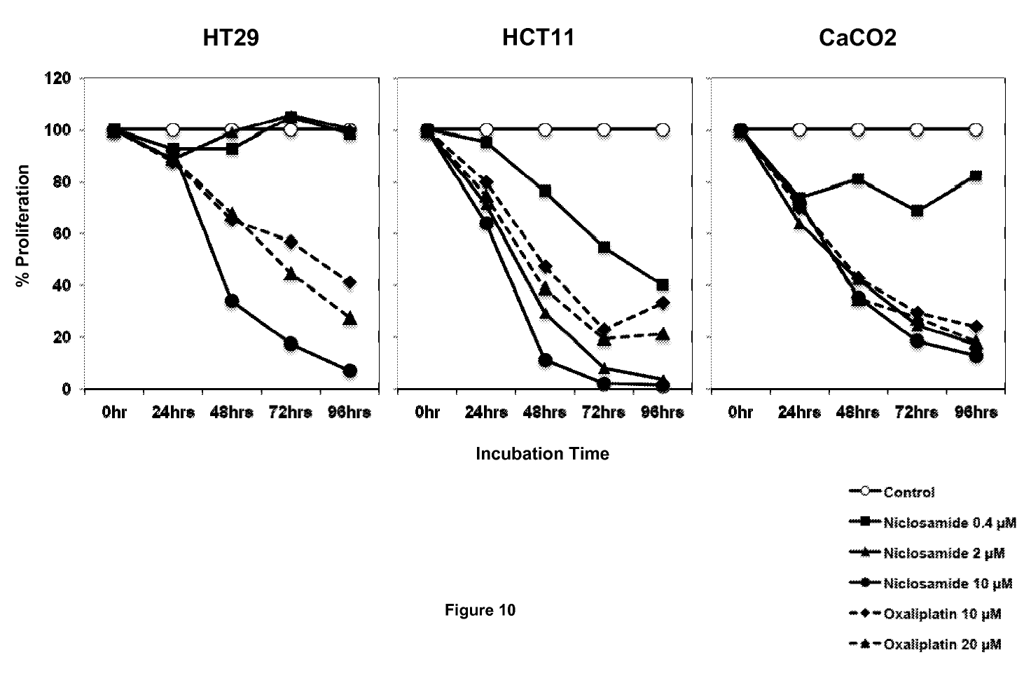

To assess the effect of niclosamide over time on colorectal cancers, 3 cell lines (HT29, HCT116, and CaCO2) were cultured at 5,000 cells per well. After overnight incubation, various concentrations of niclosamide (0.4 μM, 2 μM, 10 μM) or Oxaliplatin (10 μM, 20 μM as a positive control) were added, and the plates were incubated for 24, 48, 72, or 96 hr. MTT assay was performed at each time point. Percentage of proliferations compared with the control was determined by dividing each OD 562 nm value at the different niclosamide concentrations by OD 562 nm value of the control condition (medium alone, niclosamide 0 μM) of the same time point. As shown in FIG. 10, HT29 cells were insensitive for niclosamide, while HCT116 and CaCO2 were sensitive to niclosamide. HCT116 were killed at a lower concentration (0.4 μM) of niclosamide over time than the other cell lines. Interestingly, 2 μM and 10 μM niclosamide were more cytotoxic to the colorectal cancer cell lines than 10 μM or 20 μM of oxaliplatin, which is known to be clinically effective.

### Example 12

### Niclosamide Effect on Non-Tumor Cells

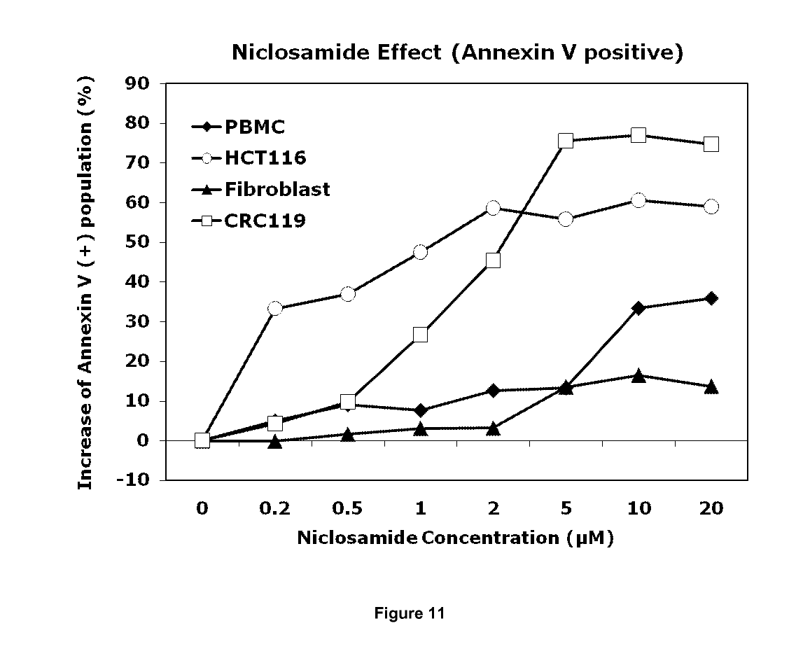

Annexin V may be used as a marker for apoptosis and other forms of cell death. To assess the toxicity of niclosamide, peripheral blood mononuclear cells (PBMCs) from a normal donor and fibroblasts isolated from colorectal cancer patient's tumor tissue were used and examined for annexin V. HCT116 and CRC119 tumor cells (1×10⁵ cells/well), fibroblasts (1×10⁵ cells/well), or peripheral blood mononuclear cells (PBMCs, 1×10⁶ cells/well) derived from normal donors, were cultured in 12 well flat bottom plates with niclosamide at concentrations ranging from 0.2 μM to 20 μM. After 3 days of incubation, all cells were harvested with 0.05% trypsin/EDTA, labeled with biotin-conjugated Annexin V, and then stained with 7-AAD and streptavidin-APC. Samples were acquired by FACSCalibur, and percentages of annexin V-positive cells were analyzed and plotted as shown in FIG. 11. Cells were analyzed for expression of Annexin V as a marker of apoptosis. The percentage increase in Annexin V-positive population (compared with untreated control) is shown for each cell type for different concentrations of niclosamide. CRC explant and HCT116 cells showed steep increase of annexin V-positive cells at 1 μM or 0.2 μM of niclosamide, respectively. Maximum percentage of annexin V-positive cells reached around 60 to 80% in these tumor cells, suggesting significant induction of apoptotic cell death. However, fibroblasts did not show a significant increase in apoptotic cell death, and PBMCs showed apoptosis at higher doses (˜10-20 μM). The results indicated that niclosamide did not have strong toxicity against non-tumor cells, while mild apoptosis may be induced in PBMCs at higher concentrations (˜10-20 μM).

### Example 13

### Niclosamide Inhibited the Cytosolic Expression of Endogenous Dishevelled-2 and 1′-catenin in Colorectal Cancer Cells

Dvl-2 and β-catenin expression by CRC057 explants and colorectal cell line HCT116 were treated with different concentrations (0, 1 μM, 5 μM, 10 μM) of niclosamide overnight (18 hr). After washing cells with PBS, cell lysates were made with hypotonic lysis buffer. Cytosolic fractions of lysates were isolated and analyzed by western blot with anti-Dishevelled 2 (clone 3F12), anti-β-catenin (clone 7D11), and anti-β-actin (clone C-11) monoclonal antibodies. It was revealed that Dvl-2 expression and β-catenin expression were also downregulated by niclosamide in these cancer cells (FIG. 12). Interestingly, HT29 cells, which were insensitive to niclosamide according to cytotoxicity assays shown above (MTT assay), showed a milder change in cytosolic expression of Dvl-2/β-catenin, while sensitive CRC explants and cell lines (CRC057, CRC119, HCT116, and CaCO2) showed more evident downregulation of Dvl-2 expression and β-catenin expression. The results revealed the correlation of cytotoxic effects with Wnt/Frizzlesl signaling inhibition.

### Example 14

### Combination Effect of Niclosamide with Anti-Cancer Drug, Oxaliplatin

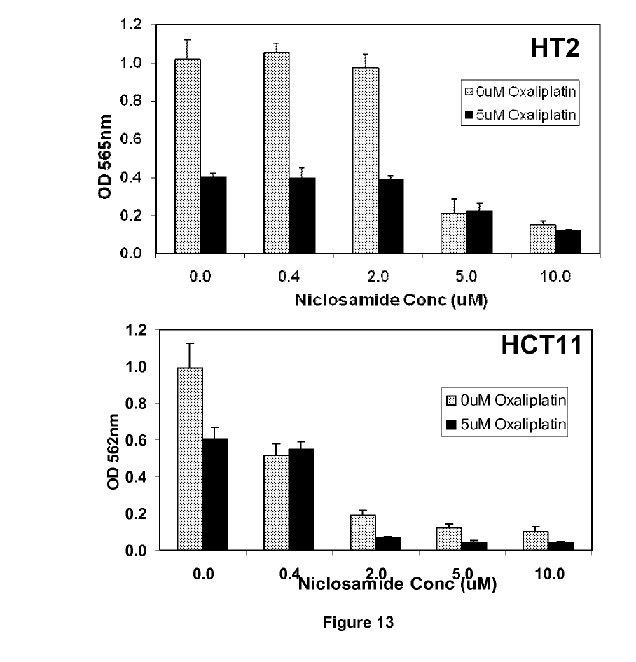

We sought to assess the combination effect of niclosamide with other anti-cancer drugs for colorectal cancers via the MTT assay after 72 hr incubation. In this study, oxaliplatin was used because it is a widely-used drug in combination therapy. Colorectal cancer cell lines (HT29, HCT116) were cultured in 96 well plates at 5,000 cells per well, and treated with various combinations of niclosamide and oxaliplatin (niclosamide at 0, 0.4 μM, 2.0 μM, 5.0 μM, 10.0 μM; oxaliplatin at 0, 5.0 μM). An MTT assay was performed after 72 hr incubation. After cell lysis with DMSO, the optical density (OD) at 562 nm was measured. As shown in FIG. 13A, addition of oxaliplatin, even at lower concentrations (˜1-2 μM), induced more killing of CaCO2 cells compared to niclosamide alone. For example, the IC50 decreased from 1.0 μM to 0.5 μM of niclosamide with the addition of 2 μM oxaliplatin. Similar additive effects were observed with CRC explant cells (CRC020, FIG. 13B), although these cells were killed significantly with 5 μM of niclosamide and additional cytotoxicity was not induced with ˜2.5-10 μM oxaliplatin.

### Example 15

### Phamacokinetic Analysis of Niclosamide Following Oral Administration in NOD/SCID Mice

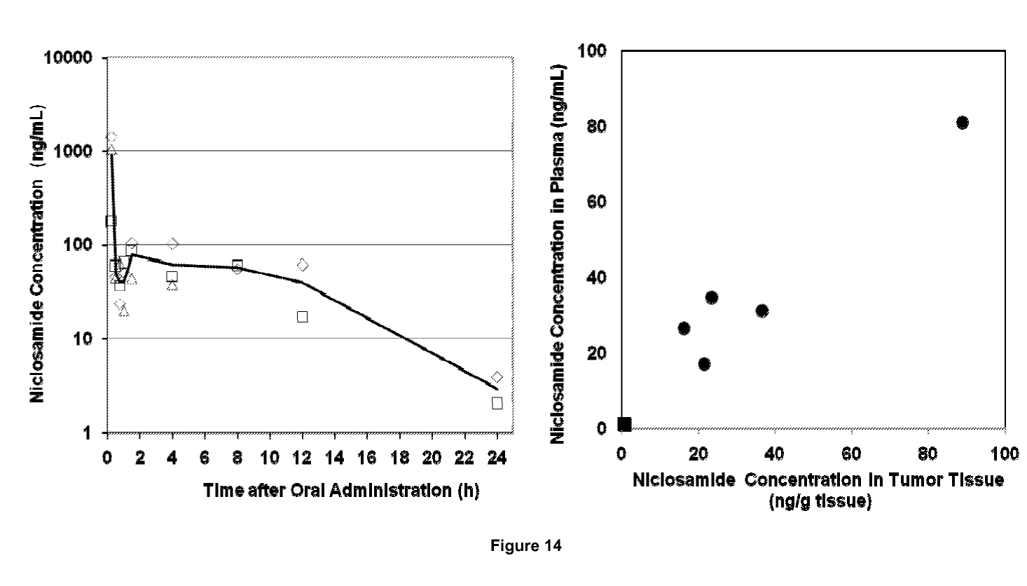

The pharmacokinetics of orally-administered niclosamide were analyzed. NOD/SCID mice (weighting 23 to 25 g) received oral administration of niclosamide (200 mg/kg of body weight in 90% Polyethylene Glycol-300, 10% 1-Methyl-2-pyrrolidone). Blood samples were obtained from vena cava at predose and at 0.25, 0.5, 0.75, 1, 1.5, 4, 8, 12, 24 hr after drug administration. Plasma was collected by the centrifugation (1,200 g, 10 min) of blood with heparin (100 U/mL). Quantification of niclosamide in mouse plasma was performed by LC/MS/MS and reported as ng/mL (based on molecular weight 327, 100 ng/mL=0.306 μM). The plasma concentrations of niclosamide in mice following oral administration of 200 mg/kg are shown in FIG. 14A. Elimination rate (z-lambda) and the half-life (t½) were calculated from the slope of 2-point line (12 and 24 hr) and were 0.217/hr and 3.2 hr. Interestingly, the concentration-time data showed a sharp peak (Tmax 0.25 hr, Cmax893.7 ng/mL) and quick decline of the plasma concentration at 30 min after oral administration, showed the second peak at 1.5 hr (77.6 ng/mL), and gradually decreased until the 24 hr time point. This re-bound at 1 hr may be explained by re-absorption of niclosamide from the intestine or distribution of the drug to the third compartment. Two-phase elimination after 1 hr could also be explained by the re-absorption. From 0.5 hr to 12 hr after oral intake, plasma concentrations were kept relatively stable in the range of 39.5 to 77.6 ng/mL (˜0.1-0.2 μM).

The niclosamide concentrations in tumor tissues were also analyzed. NOD/SCID mice were inoculated with HCT116 tumor cells (5×10⁶ cells), and on day 4, oral gavage of niclosamide (200 mg/kg body weight) or control solvent was initiated. After three weeks of treatment, mice were sacrificed 24 hr after the last oral administration, and blood and tumor tissue were collected simultaneously to measure the niclosamide concentration. Plasma was isolated from blood as described above. Tumor tissue was cryo-crushed in liquid nitrogen and homogenated with three volumes of deionized water. Quantification of niclosamide in mouse plasma and tumor tissue was performed by LC/MS/MS. Niclosamide concentrations in tumor tissue (ng/g tissue) and those in plasma (ng/mL) were plotted for each mouse (FIG. 14B). Niclosamide concentrations in tumor tissue and those in plasma showed a good correlation, suggesting the efficient distribution of niclosamide from blood to tumor tissue.

### Example 16

### Anti-Tumor Effect of Niclosamide In Vivo

As shown above, niclosamide was cytotoxic against colorectal cancer cells in in vitro assays. In vivo assays were also performed to assess anti-tumor effects of niclosamide. HCT116 cells and CRC039 cells were used.

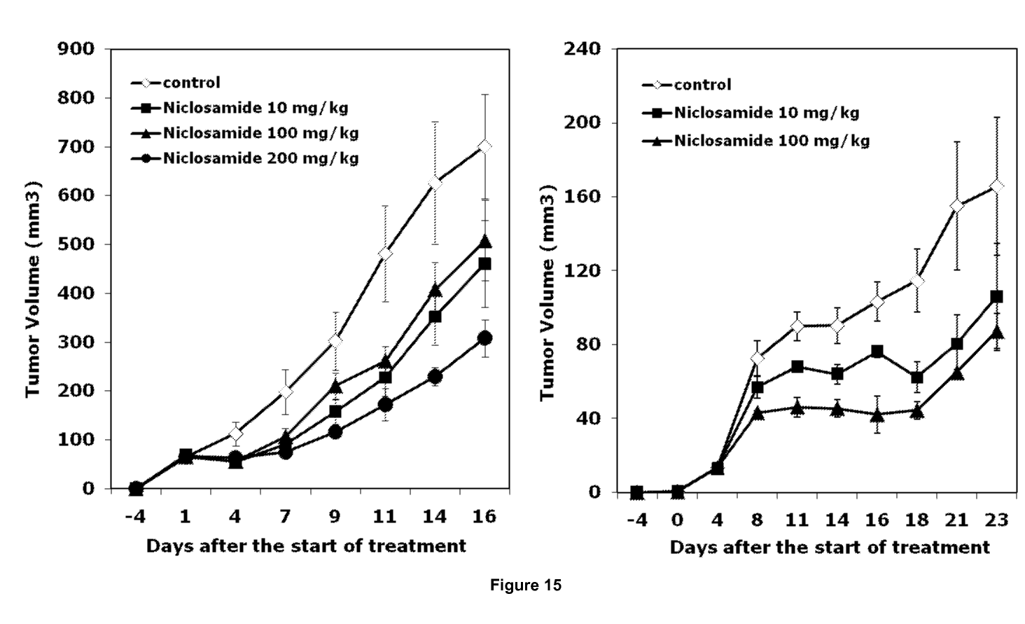

HCT116 colon cancer cells were harvested from flasks with 0.05% Trypsin/EDTA and resuspended with Hanks buffered solution at 5×10⁶ cells/100 μL concentration. CRC explants (CRC039) cultured in vitro were harvested with the same procedure and mixed with equal volume of Matrigel to make 1×10⁶ cells/100 μL concentration. The cell suspension (100 μL) was inoculated into the flank of NOD/SCID mice 4 days before the start of treatment. Niclosamide (0, 10, 100, or 200 mg/kg body weight) was administered by gavage for 6 days per week for 2 weeks (HCT116, left) or 3 weeks (CRC039, right). Initially, 2 weeks of treatment was planned for both HCT116 and CRC039. However, treatment was extended to 3 weeks for CRC039 cells because the NOD/SCID mice grew much slower than the HCT116 cell line. Tumor size was measured 3 times a week until mice were euthanized. During the course of the treatment with niclosamide, no obvious side effect was observed in the mice. As shown in FIG. 15, niclosamide significantly inhibited the growth of both HCT116 and CRC039 tumors. In fast growing tumors (HCT116), a dose of 200 mg/kg of body weight suppressed the tumor growth to less than half a volume after two weeks of treatment. However, 100 mg/kg of niclosamide could suppress the growth of relatively slow-growing tumors (CRC039) to the same extent. Another CRC explant (CRC028) was analyzed, and a similar inhibition of tumor growth with niclosamide (25 mg/kg per day) compared to non-treated mice was observed. Thus, in vitro results were confirmed in vivo, i.e., that niclosamide inhibited the growth of colorectal cancer in NOD/SCID mice.

### Example 17

### Dishevelled-2 and 1′-catenin Expression in Niclosamide Treated Colorectal Cancer Tumors

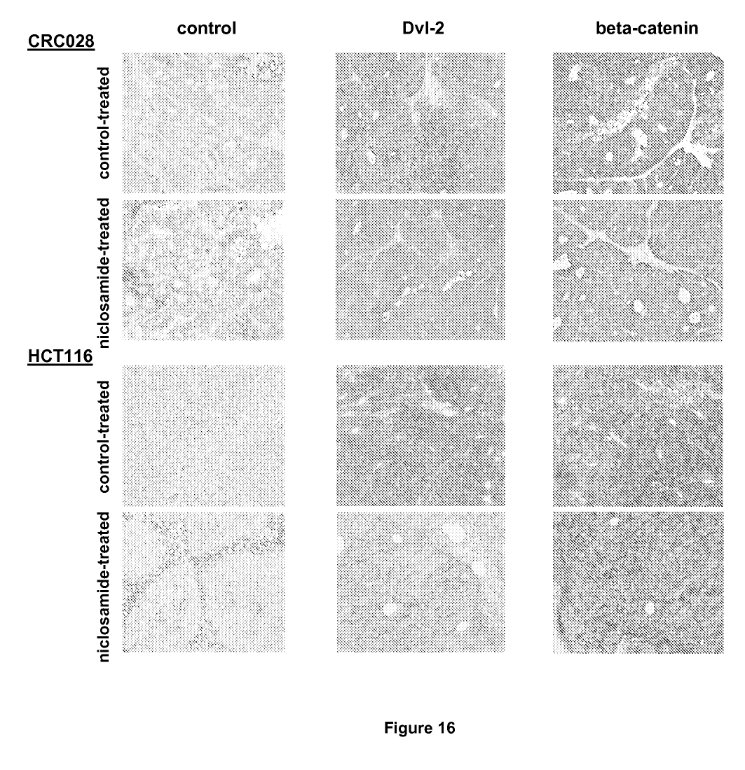

NOD/SCID mice were implanted with colorectal cancer explants or cell lines. After the establishment of tumors in the flank, mice were treated with/without niclosamide (200 mg/kg body weight) for 2 or 3 weeks. Tumors were excised and fixed with 10% neutralized, buffered formalin. Paraffin-embedded specimens were cut into 4 μm-thick of sequential sections. After dewaxing and rehydration, heat-induced antigen retrieval was performed. Endogenous peroxidase activity was blocked with 3% H₂O₂, and non-specific binding was blocked with non-immune horse sera. Sections were then incubated with anti-β-catenin and anti-Dvl-2 monoclonal antibodies (1:50 dilution) overnight at 4° C. After rinsing, the sections were incubated with the biotinylated secondary antibody (1:200 dilution) for 30 min at room temperature and then with the avidin-biotin peroxidase complex. Visualization was achieved using 3,3′-diaminobenzidine tetrahydrochloride and H₂O₂. Specimens were counterstained with hematoxylin. Niclosamide treated tumors showed decreased levels of cytoplasmic expression of Dvl-2 and β-catenin compared to control treated tumors (FIG. 16, magnification at 200×). This result indicates the prolonged inhibitory effect of niclosamide in vivo for Wnt/β-catenin signaling.

### Example 18

### Niclosamide Compounds Treat Hypertension

In vivo assays are performed to assess the effects of niclosamide compounds on cardiovascular diseases, such as hypertension. The assays will use existing or later developed models of one or more various cardiovascular diseases such as, for example, rat models of cardiovascular disease, such as Spontaneously Hypertensive Rats (SHR/NCrI, Charles River Laboratories, Germantown, Md.). The model system can incorporate a dysregulated Wnt/Frizzled signaling cascade. A niclosamide compound is administered by any acceptable route (e.g., orally, transmucousally, via injection, etc.) and by any known technique, such as, by gavage technique. Upon administration of the niclosamide compound in therapeutically relevant amounts, (for example, at 0, 10, 100, or 200 mg/kg 6 times a week, for 1-10 weeks) the niclosamide compounds will be expected to show a significant reduction and/or improvement in the physiological symptoms and/or the biological markers that are indicative of hypertension in the model system.

### Example 19

### Niclosamide Compounds Treat Cardiovascular Disease

In vivo assays are performed to assess the effects of niclosamide compounds on cardiovascular disease. Cardiovascular disease rats, such as Spontaneously Hypertensive Heart Failure Rats (SHHF/MccGmiCrI-Lepr^{cp}, Charles River Laboratories, Germantown, Md.), are used. The model system can incorporate a dysregulated Wnt/Frizzled signaling cascade. A niclosamide compound is administered by any acceptable route (e.g., orally, transmucousally, via injection, etc.) and by any known technique, such as by gavage technique. Upon administration of the niclosamide compound in therapeutically relevant amounts, (for example, at 0, 10, 100, or 200 mg/kg 6 times a week, for 1-10 weeks) the niclosamide compounds will be expected to show a significant reduction and/or improvement in the physiological symptoms and/or the biological markers that are indicative of heart failure.

### Example 20

### Effect of Niclosamide Derivatives or Analogs on Wnt/Frizzled Signaling Pathway

The compounds shown in Table 2 were purchased from Sigma-Aldrich (St. Louis, Mo.) or Chembridge (San Diego, Calif.). The activity of each of the compounds as a negative modulator of the Wnt/Frizzled signaling pathway was examined using the Top-flash assay, as described above. Results are presented in Table 3.

TABLE 2
Niclosamide compounds.
I

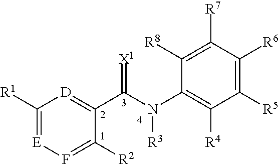

X¹ = O, D = CR⁹; E = CR¹⁰; F = CR¹¹; R = H unless designated below

| # | Compound | MW | R¹ | R² | R³ | R⁴ | R⁵ | R⁶ | R⁷ | R⁸ | R⁹ | R¹⁰ | R¹¹ |
|---|---|---|---|---|---|---|---|---|---|---|---|---|---|
| 1-2 | 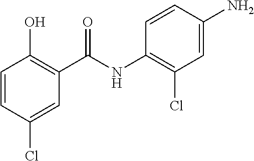 N-(4-amino-2-chlorophenyl)-5- chloro-2-hydroxybenzamide | 297.14 | H | H | H | Cl | H | NH₂ | H | H | OH | H | Cl |
| 1-4 | 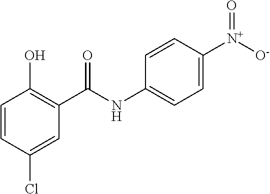 5-chloro-2-hydroxy-N-(4- nitrophenyl)benzamide | 292.68 | H | H | H | H | H | NO₂ | H | H | OH | H | Cl |
| 1-5 | 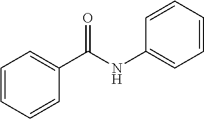 benzanilide, 98% | 197.24 | H | H | H | H | H | H | H | H | H | H | H |
| 1-6 | 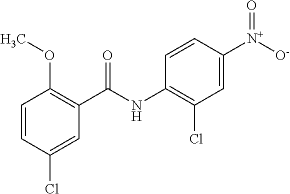 Benzamide, 5-chloro-N-(2-chloro-4- nitrophenyl)-2-methoxy- | 341.15 | H | H | H | Cl | H | NO₂ | H | H | OCH₃ | H | Cl |
| 1-7 | 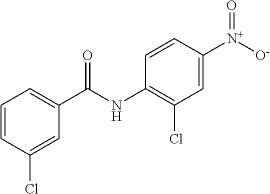 Benzamide, 3-chloro-N-(2-chloro-4- nitrophenyl)- | 311.13 | H | H | H | Cl | H | NO₂ | H | H | H | H | Cl |
| 1-8 | 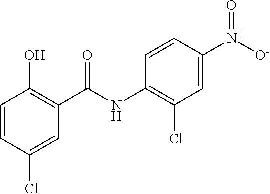 niclosamide | 327.13 | H | H | H | Cl | H | NO₂ | H | H | OH | H | Cl |
| 2-1 | 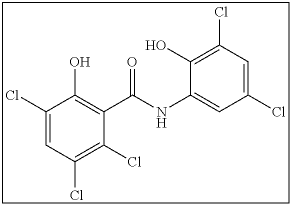 Oxyclozanide | 401.46 | Cl | Cl | H | H | Cl | H | Cl | OH | OH | H | Cl |
| 2-2 | 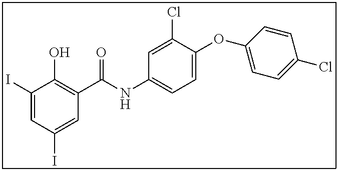 Rafoxanide | 626.01 | I | H | H | H | H | O-(4-Cl—Ph) | Cl | H | OH | H | I |
| 2-3 | 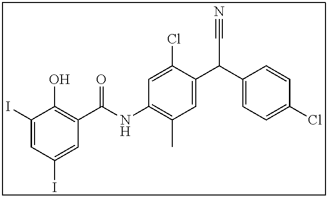 Closantel | 663.07 | I | H | H | CH₃ | H | (4- CH(CN)—ClPh) | Cl | H | OH | H | I |
| 2-4 | 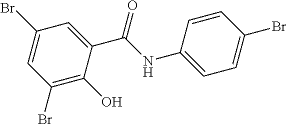 3,5-dibromo-N-(4-bromophenyl)- 2- hydroxybenzamide | 449.9 | Br | OH | H | H | H | Br | H | H | H | H | Br |
| | 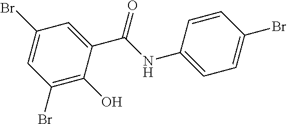 [SMILES: O=C(Nc1ccc(Br)cc1)c1cc(Br)cc(Br)c1O] 3,5-dibromo-N-(4-bromophenyl)-2- hydroxybenzamide | | | | | | | | | | | | |
| 3-1 | 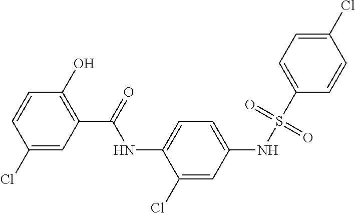 5-chloro-N-(2-chloro-4-{[(4- chlorophenyl)sulfonyl]amino}phenyl)- 2-hydroxybenzamide | 471.7 | H | H | H | Cl | H | NHSO₂- (4-Cl—Ph) | H | H | OH | H | Cl |
| 3-2 | 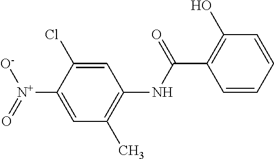 N-(5-chloro-2-methyl-4-nitrophenyl)- 2-hydroxybenzamide | 306.7 | H | OH | H | H | Cl | NO₂ | H | CH₃ | H | H | H |
| 3-3 | 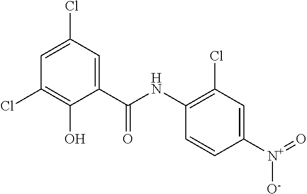 3,5-dichloro-N-(2-chloro-4- nitrophenyl)-2-hydroxybenzamide | 361.6 | Cl | OH | H | H | H | NO₂ | H | Cl | H | H | Cl |
| 3-4 | 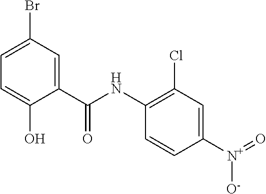 5-bromo-N-(2-chloro-4-nitrophenyl)- 2-hydroxybenzamide | 371.6 | Br | OH | H | H | H | NO₂ | H | Cl | H | H | H |
| 3-5 | 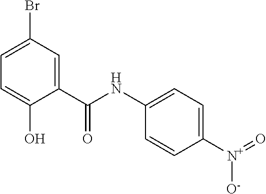 5-bromo-2-hydroxy-N-(4- nitrophenyl)benzamide | 337.1 | Br | OH | H | H | H | NO₂ | H | H | H | H | H |
| 3-6 | 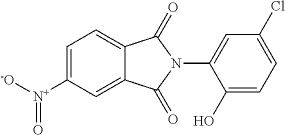 2-(5-chloro-2-hydroxyphenyl)-5- nitro-1H-isoindole-1,3(2H)-dione | 318.7 | | | | | | | | | | | |
| 3-7 | 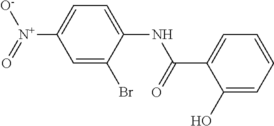 N-(2-bromo-4-nitrophenyl)-2- hydroxybenzamide | 337.1 | H | H | H | H | H | NO₂ | H | Br | OH | H | H |
| 3-8 | 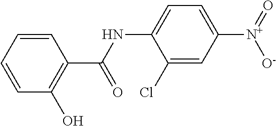 N-(2-chloro-4-nitrophenyl)-2- hydroxybenzamide | 292.7 | H | OH | H | Cl | H | NO₂ | H | H | H | H | H |
| 3-9 | 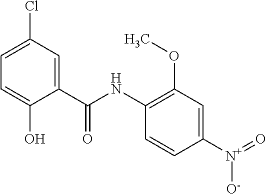 5-chloro-2-hydroxy-N-(2-methoxy-4- nitrophenyl)benzamide | 322.7 | Cl | OH | H | H | H | NO₂ | H | OCH₃ | H | H | H |
| 3-10 | 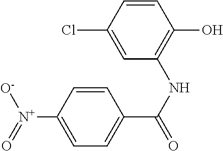 [SMILES: O=C(Nc1cc(Cl)ccc1O)c1ccc([N+](=O)[O-])cc1] N-(5-chloro-2-hydroxyphenyl)-4- nitrobenzamide | 292.7 | | | | | | | | | | | |
| 3-11 | 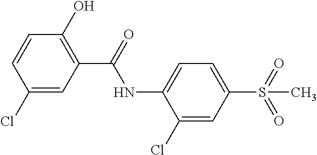 5-chloro-N-[2-chloro-4- (methylsulfonyl)phenyl]-2- hydroxybenzamide | 360.2 | H | H | H | Cl | H | SO₂CH₃ | H | H | OH | H | Cl |

TABLE 3
Activity of niclosamide compounds as negative modulators of the
Wnt/Frizzled signaling pathway.

| | | | TopFlash assay: |
|---|---|---|---|
| | | Primary assay | IC₅₀ < 1 μM (+++), |
| | | at 12.5 μM: | 1 μm < IC₅₀ < 12.5 μM (++), |
| | | A = active, | IC₅₀ > 12.5 μM (+), |
| | | WA = weak active, | no measurable |
| | | I = inactive, | inhibitory activity at |
| # | Compound | NE = not evaluated | 12.5 μM (−) |
| 1-2 | 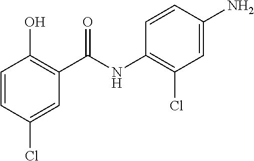 [SMILES: Nc1ccc(NC(=O)c2cc(Cl)ccc2O)c(Cl)c1] N-(4-amino-2-chlorophenyl)-5- chloro-2-hydroxybenzamide | WA | + |
| 1-4 | 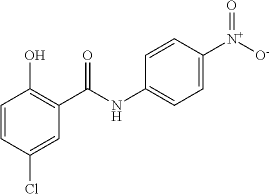 [SMILES: O=C(Nc1ccc([N+](=O)[O-])cc1)c1cc(Cl)ccc1O] 5-chloro-2-hydroxy-N-(4- nitrophenyl)benzamide | A | +++ |
| 1-5 | 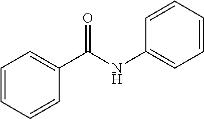 [SMILES: O=C(Nc1ccccc1)c1ccccc1] benzanilide, 98% | I | − |
| 1-6 | 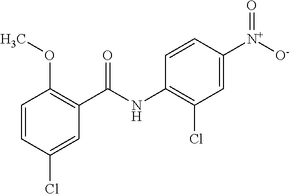 [SMILES: COc1ccc(Cl)cc1C(=O)Nc1ccc([N+](=O)[O-])cc1Cl] Benzamide, 5-chloro-N-(2-chloro-4- nitrophenyl)-2-methoxy- | I | + |
| 1-7 | 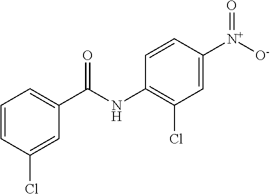 [SMILES: O=C(Nc1ccc([N+](=O)[O-])cc1Cl)c1cccc(Cl)c1] Benzamide, 3-chloro-N-(2-chloro-4- nitrophenyl)- | I | − |
| 1-8 | 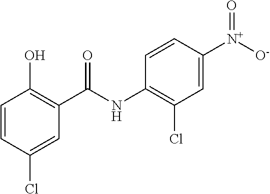 [SMILES: O=C(Nc1ccc([N+](=O)[O-])cc1Cl)c1cc(Cl)ccc1O] niclosamide | A | +++ |
| 2-1 | 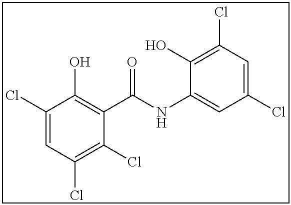 [SMILES: O=C(Nc1cc(Cl)cc(Cl)c1O)c1c(O)c(Cl)cc(Cl)c1Cl] Oxyclozanide | WA | + |
| 2-2 | 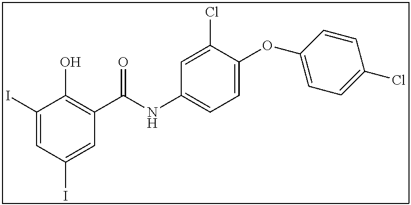 [SMILES: O=C(Nc1ccc(Oc2ccc(Cl)cc2)c(Cl)c1)c1cc(I)cc(I)c1O] Rafoxanide | WA | + |
| 2-3 | 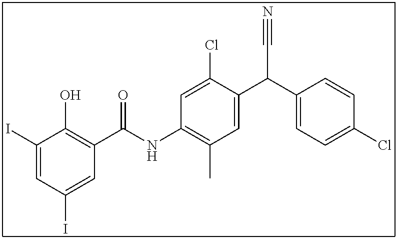 [SMILES: Cc1cc(C(C#N)c2ccc(Cl)cc2)c(Cl)cc1NC(=O)c1cc(I)cc(I)c1O] Closantel | WA | + |
| 2-4 | 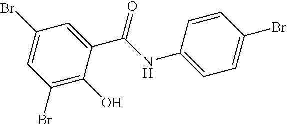 [SMILES: O=C(Nc1ccc(Br)cc1)c1cc(Br)cc(Br)c1O] 3,5-dibromo-N-(4-bromophenyl)-2- hydroxybenzamide | WA | + |
| | 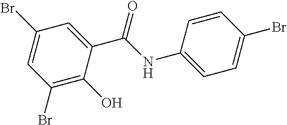 [SMILES: O=C(Nc1ccc(Br)cc1)c1cc(Br)cc(Br)c1O] 3,5-dibromo-N-(4-bromophenyl)-2- hydroxybenzamide | | |
| 3-1 | 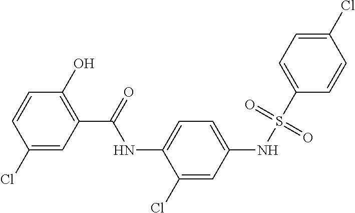 [SMILES: O=C(Nc1ccc(NS(=O)(=O)c2ccc(Cl)cc2)cc1Cl)c1cc(Cl)ccc1O] 5-chloro-N-(2-chloro-4-{[(4- chlorophenyl)sulfonyl]amino}phenyl)- 2-hydroxybenzamide | I | − |
| 3-2 | 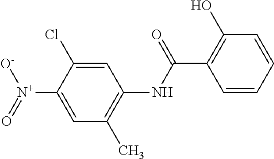 [SMILES: Cc1cc([N+](=O)[O-])c(Cl)cc1NC(=O)c1ccccc1O] N-(5-chloro-2-methyl-4-nitrophenyl)- 2-hydroxybenzamide | A | +++ |
| 3-3 | 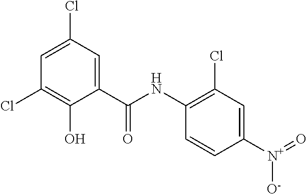 [SMILES: O=C(Nc1ccc([N+](=O)[O-])cc1Cl)c1cc(Cl)cc(Cl)c1O] 3,5-dichloro-N-(2-chloro-4- nitrophenyl)-2-hydroxybenzamide | A | ++ |
| 3-4 | 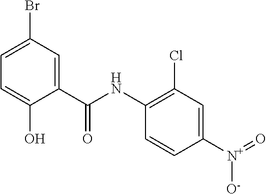 [SMILES: O=C(Nc1ccc([N+](=O)[O-])cc1Cl)c1cc(Br)ccc1O] 5-bromo-N-(2-chloro-4-nitrophenyl)- 2-hydroxybenzamide | A | +++ |
| 3-5 | 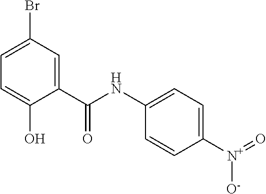 [SMILES: O=C(Nc1ccc([N+](=O)[O-])cc1)c1cc(Br)ccc1O] 5-bromo-2-hydroxy-N-(4- nitrophenyl)benzamide | A | +++ |
| 3-6 | 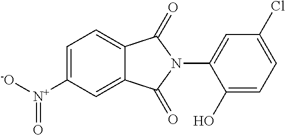 2-(5-chloro-2-hydroxyphenyl)-5-nitro- 1H-isoindole-1,3(2H)-dione | I | − |
| 3-7 | 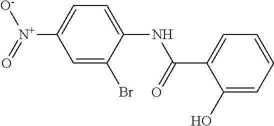 [SMILES: O=C(Nc1ccc([N+](=O)[O-])cc1Br)c1ccccc1O] N-(2-bromo-4-nitrophenyl)-2- hydroxybenzamide | A | ++ |
| 3-8 | 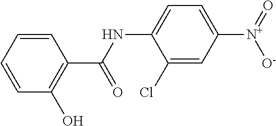 [SMILES: O=C(Nc1ccc([N+](=O)[O-])cc1Cl)c1ccccc1O] N-(2-chloro-4-nitrophenyl)-2- hydroxybenzamide | A | ++ |
| 3-9 | 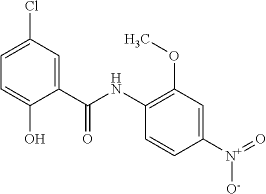 [SMILES: COc1cc([N+](=O)[O-])ccc1NC(=O)c1cc(Cl)ccc1O] 5-chloro-2-hydroxy-N-(2-methoxy-4- nitrophenyl)benzamide | A | +++ |
| 3-10 | 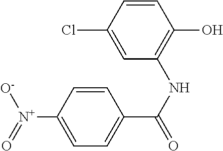 [SMILES: O=C(Nc1cc(Cl)ccc1O)c1ccc([N+](=O)[O-])cc1] N-(5-chloro-2-hydroxyphenyl)-4- nitrobenzamide | I | − |
| 3-11 | 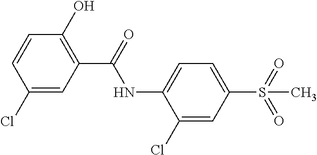 [SMILES: CS(=O)(=O)c1ccc(NC(=O)c2cc(Cl)ccc2O)c(Cl)c1] 5-chloro-N-[2-chloro-4- (methylsulfonyl)phenyl]-2- hydroxybenzamide | I | + |

| Wnt3A = | |
|---|---|
| SEQ ID NO: 1 | |
| polypeptide sequence | |
| homo sapiens, accession number BAB61052 | |
| 352 amino acids | |
| 1 | maplgyflll cslkqalgsy piwwslavgp qysslgsqpi lcasipglvp kqlrfcrnyv |
| 61 | eimpsvaegi kigiqecqhq frgrrwnctt vhdslaifgp vldkatresa fvhaiasagv |
| 121 | afavtrscae gtaaicgcss rhqgspgkgw kwggcsedie fggmvsrefa darenrpdar |
| 181 | samnrhnnea grqaiashmh lkckchglsg scevktcwws qpdfraigdf lkdkydsase |
| 241 | mvvekhresr gwvetlrpry tyfkvpterd lvyyeaspnf cepnpetgsf gtrdrtcnvs |
| 301 | shgidgcdll ccgrghnara errrekcrcv fhwccyvscq ectrvydvht ck |
| β-catenin = | |
| SEQ ID NO: 2 | |
| polypeptide sequence | |
| homo sapiens, accession number NP_001091679, NP_001091680.1, or | |
| NP_001895.1 781 amino acids | |
| 1 | matqadlmel dmamepdrka ayshwqqqsy ldsgihsgat ttapslsgkg npeeedvdts |
| 61 | qvlyeweqgf sqsftqeqva didgqyamtr aqrvraamfp etldegmqip stqfdaahpt |
| 121 | nvqrlaepsq mlkhavvnli nyqddaelat raipeltkll ndedqvvvnk aavmvhqlsk |
| 181 | keasrhaimr spqmvsaivr tmqntndvet arctaqtlhn lshhreqlla ifksqqipal |
| 241 | vkmlgspvds vlfyaittlh nlllhqegak mavrlagglq kmvallnktn vkflaittdc |
| 301 | lqilaygnqe skliilasgg pqalvnimrt ytyekllwtt srvlkvlsvc ssnkpaivea |
| 361 | ggmqalglhl tdpsqrlvqn clwtlrnlsd aatkqegmeg llgtlvqllg sddinvvtca |
| 421 | agilsnltcn nyknkmmvcq vggiealvrt vlragdredi tepaicalrh ltsrhqeaem |
| 481 | aqnavrlhyg lpvvvkllhp pshwplikat vglirnlalc panhaplreq gaiprlvqll |
| 541 | vrahqdtqrr tsmggtqqqf vegvrmeeiv egctgalhil ardvhnrivi rglntiplfv |
| 601 | qllyspieni qrvaagvlce laqdkeaaea ieaegatapl tellhsrneg vatyaaavlf |
| 661 | rmsedkpqdy kkrlsvelts slfrtepmaw netadlgldi gaqgeplgyr qddpsyrsfh |
| 721 | sggygqdalg mdpmmehemg ghhpgadypv dglpdlghaq dlmdglppgd snqlawfdtd |
| 781 | l |
| Frizzled1 = | |
| SEQ ID NO: 3 | |
| polypeptide sequence | |
| homo sapiens, accession number NP_003496.1 | |
| 647 amino acids | |
| 1 | maeeeapkks raagggaswe lcagalsarl aeegsgdagg rrrppvdprr larqlllllw |
| 61 | lleaplllgv raqaagqgpg qgpgpgqqpp pppqqqqsgq qyngergisv pdhgycqpis |
| 121 | iplctdiayn qtimpnllgh tnqedaglev hqfyplvkvq csaelkfflc smyapvctvl |
| 181 | eqalppcrsl cerarqgcea lmnkfgfqwp dtlkcekfpv hgagelcvgq ntsdkgtptp |
| 241 | sllpefwtsn pqhgggghrg gfpggagase rgkfscpral kvpsylnyhf lgekdcgapc |
| 301 | eptkvyglmy fgpeelrfsr twigiwsvlc castlftvlt ylvdmrrfsy perpiiflsg |
| 361 | cytavavayi agflledrvv cndkfaedga rtvaqgtkke gctilfmmly ffsmassiww |
| 421 | vilsltwfla agmkwgheai eansqyfhla awavpaikti tilalgqvdg dvlsgvcfvg |
| 481 | lnnvdalrgf vlaplfvylf igtsfllagf vslfrirtim khdgtktekl eklmvrigvf |
| 541 | svlytvpati viacyfyeqa frdqwerswv aqscksyaip cphlgaggga pphppmspdf |
| 601 | tvfmikylmt livgitsgfw iwsgktlnsw rkfytrltns kqgettv |
| Dishevelled Dvl1 = | |
| SEQ ID NO: 4 | |
| polypeptide sequence | |
| homo sapiens, accession number CAI23185.1 | |
| 670 amino acids | |
| 1 | maetkiiyhm deeetpylvk lpvapervtl adfknvlsnr pvhaykfffk smdqdfgvvk |
| 61 | eeifddnakl pcfngrvvsw lvlaegahsd agsqgtdsht dlppplertg gigdsrppsf |
| 121 | hpnvassrdg mdnetgtesm vshrrerarr rnreeaartn ghprgdrrrd vglppdsast |
| 181 | alsselesss fvdsdedgst srlsssteqs tssrlirkhk rrrrkqrlrq adrassfssi |
| 241 | tdstmslniv tvtlnmerhh flgisivgqs ndrgdggiyi gsimkggava adgriepgdm |
| 301 | llqvndvnfe nmsnddavrv lreivsqtgp isltvakcwd ptprsyftvp radpvrpidp |
| 361 | aawlshtaal tgalpryele eapltvksdm savvrvmqlp dsgleirdrm wlkitianav |
| 421 | igadvvdwly thvegfkerr earkyassll khgflrhtvn kitfseqcyy vfgdlcsnla |
| 481 | tlnlnsgssg tsdqdtlapl phpaapwplg qgypyqypgp ppcfppayqd pgfsygsgst |
| 541 | gsqqsegsks sgstrssrra pgrekerraa gaggsgsesd htapsgvgss wrerpagqls |
| 601 | rgssprsqas atapglppph pttkaytvvg gppggppvre laavppeltg srqsfqkamg |
| 661 | npceffvdim |
| Dishevelled Dvl2 = | |
| SEQ ID NO: 5 | |
| polypeptide sequence | |
| homo sapiens, accession number NP_004413.1 | |
| 736 amino acids | |
| 1 | magsstgggg vgetkviyhl deeetpylvk ipvpaeritl gdfksvlqrp agakyffksm |
| 61 | dqdfgvvkee isddnarlpc fngrvvswlv ssdnpqpema ppvheprael appapplppl |
| 121 | ppertsgigd srppsfhpnv ssshenlepe tetesvvslr rerprrrdss ehgagghrtg |
| 181 | gpsrlerhla gyessstlmt selestslgd sdeedtmsrf sssteqssas rllkrhrrrr |
| 241 | kqrpprlert ssfssvtdst mslniitvtl nmekynflgi sivgqsnerg dggiyigsim |
| 301 | kggavaadgr iepgdmllqv ndmnfenmsn ddavrvlrdi vhkpgpivlt vakcwdpspq |
| 361 | ayftlprnep iqpidpaawv shsaaltgtf paypgsssms titsgsslpd gcegrglsvh |
| 421 | tdmasvtkam aapesglevr drmwlkitip naflgsdvvd wlyhhvegfp errearkyas |
| 481 | gllkaglirh tvnkitfseq cyyvfgdlsg gcesylvnls lndndgssga sdqdtlaplp |
| 541 | gatpwpllpt fsyqypaphp yspqpppyhe lssytygggs assqhsegsr ssgstrsdgg |
| 601 | agrtgrpeer apesksgsgs esepssrggs lrrggeasgt sdggpppsrg stggapnlra |
| 661 | hpglhpygpp pgmalpynpm mvvmmppppp pvppavqppg appvrdlgsv ppeltasrqs |
| 721 | fhmamgnpse ffvdvm |
| Dishevelled Dvl3 = | |
| SEQ ID NO: 6 | |
| polypeptide sequence | |
| homo sapiens, accession number NP_004414.3 | |
| 716 amino acids | |
| 1 | mgetkiiyhl dggetpylvk lplpaervtl adfkgvlqrp sykfffksmd ddfgvvkeei |
| 61 | sddnaklpcf ngrvvswlvs aegshpdpap fcadnpselp ppmertggig dsrppsfhph |
| 121 | agggsqenld ndtetdslvs aqrerprrrd gpehatrlng takgerrrep ggydssstlm |
| 181 | sselettsff dsdeddstsr fsssteqssa srlmrrhkrr rrkqkvsrie rsssfssitd |
| 241 | stmslniitv tlnmekynfl gisivgqsne rgdggiyigs imkggavaad griepgdmll |
| 301 | qvneinfenm snddavrvlr eivhkpgpit ltvakcwdps prgcftlprs epirpidpaa |
| 361 | wvshtaamtg tfpaygmsps lstitstsss itssipdter lddfhlsihs dmaaivkama |
| 421 | spesglevrd rmwlkitipn afigsdvvdw lyhnvegftd rrearkyasn llkagfirht |
| 481 | vnkitfseqc yyifgdlcgn manlslhdhd gssgasdqdt laplphpgaa pwpmafpyqy |
| 541 | pppphpynph pgfpelgysy gggsassqhs egsrssgsnr sgsdrrkekd pkagdsksgg |
| 601 | sgsesdhttr sslrgprera psersgpaas ehshrshhsl asslrshhth psygppgvpp |
| 661 | lygppmlmmp pppaamgppg appgrdlasv ppeltasrqs frmamgnpse ffvdvm |

1. A method of treating a Wnt/Frizzled-related disease in a subject in need of such treatment, the method comprising administering to the subject an effective amount of a niclosamide compound, or pharmaceutically acceptable salt thereof, of Formula I: 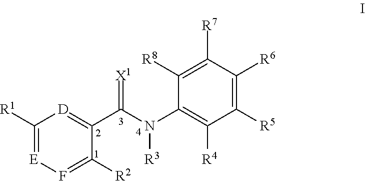
wherein
D is N or CR⁹;
E is N or CR¹⁰;
F is N or CR¹¹;
R¹ is H, halide, OR¹², SR¹³NR¹⁴R¹⁵, or described by one of the formulas: 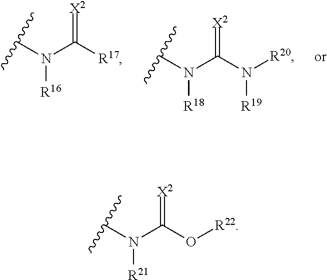
R² is H, OH, or OR¹²;
R³ is H, C₁₋₇ alkyl, C₂₋₇ alkenyl, C₂₋₇ alkynyl, C₂₋₆ heterocyclyl, C₆₋₁₂ aryl, C₇₋₁₄ alkylaryl, C₃₋₁₀ alkylheterocyclyl, or C₁₋₇ heteroalkyl;
or R² and R³ combine to form a six-membered ring in which position 1 is connected to position 4 by one of the groups: 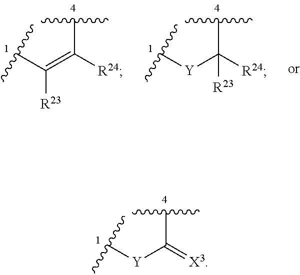
R⁴ and R⁸ are each, independently, selected from H, halide, CF₃, OR²⁸, C₁₋₇ alkyl, C₂₋₇ alkenyl, C₂₋₇ alkynyl, C₂₋₆ heterocyclyl, C₇₋₁₄ alkaryl, C₃₋₁₀ alkheterocyclyl, or C₁₋₇ heteroalkyl;
R⁵, R⁶, and R⁷ are each, independently, selected from H, C₁₋₇ alkyl, C₂₋₇ alkenyl, C₂₋₇ alkynyl, C₂₋₆ heterocyclyl, C₆₋₁₂ aryl, C₇₋₁₄ alkaryl, C₃₋₁₀ alkheterocyclyl, or C₁₋₇ heteroalkyl, halide, NO₂, CO₂H, SO₃H, CF₃, CN, OR²⁹, SR³⁰, or are described by the formulas: 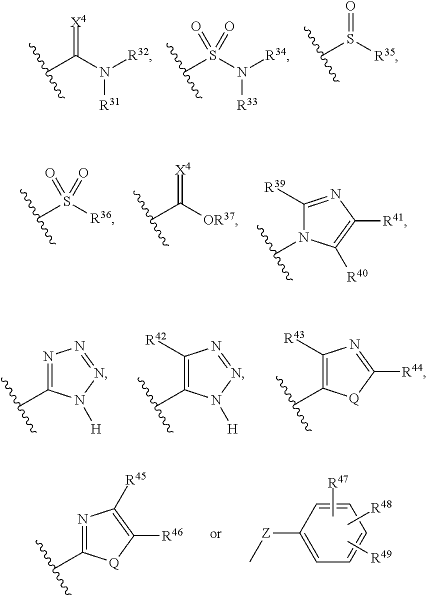
X¹, X², X³, and X⁴ is, independently, O, S; or NR³⁸;
Y is CR²⁵R²⁶, O, S, or NR²⁷;
Z is O, S, or CR⁵⁰R⁵¹;
Q is, independently, O, S, or NR⁵²;
R⁹, R¹⁰, and R¹¹ are each, independently, H, OH, OR¹², C₁₋₇ alkyl, C₂₋₇ alkenyl, C₂₋₇ alkynyl, C₁₋₇ heteroalkyl, halide, or NO₂;
R¹² and R¹³ are each, independently, acyl, C₁₋₇ alkyl, C₂₋₇ alkenyl, C₂₋₇-alkynyl, C₂₋₆ heterocyclyl, C₆₋₁₂ aryl, C₇₋₁₄ alkaryl, C₃₋₁₀ alkheterocyclyl, or C₁₋₇ heteroalkyl;
R¹⁷, R²², R³⁵, R³⁶, R³⁷, R³⁸ and R⁵² are each, independently, C₁₋₇ alkyl, C₂₋₇ alkenyl, C₂₋₇ alkynyl, C₂₋₆ heterocyclyl, C₆₋₁₂ aryl, C₇₋₁₄ alkaryl, C₃₋₁₀ alkheterocyclyl, or C₁₋₇ heteroalkyl;
R¹⁴, R¹⁵, R¹⁶, R¹⁸, R¹⁹, R²⁰, R²¹, R²³, R²⁴, R²⁵, R²⁶, R²⁷, R²⁸, R²⁹, R³⁰, R³¹, R³², R³³, R³⁴, and R⁴⁷ are each, independently, H, C₁₋₇ alkyl, C₂₋₇ alkenyl, C₂₋₇alkynyl, C₂₋₆ heterocyclyl, C₆₋₁₂ aryl, C₇₋₁₄ alkaryl, C₃₋₁₀ alkheterocyclyl, or C₁₋₇ heteroalkyl; and
R³⁹, R⁴⁰, R⁴¹, R⁴², R⁴³, R⁴⁴, R⁴⁵, R⁴⁶, R⁴⁷, R⁴⁸, R⁴⁹, R⁵⁰, and R⁵¹ are each, independently, H, halide, CN, NO₂, CF₃, C₁₋₇ alkyl, C₂₋₇ alkenyl, C₂₋₇ alkynyl, C₂₋₆ heterocyclyl, C₆₋₁₂ aryl, C₇₋₁₄ alkaryl, C₃₋₁₀ alkheterocyclyl, or C₁₋₇ heteroalkyl,
wherein the Wnt/Frizzled-related disease is not a neoplasm.

2. The method of claim 1, wherein the Wnt/Frizzled-related disease is a cardiovascular disease.

3. A method of treating a Wnt/Frizzled-related disease in a subject comprising:
a) identifying a subject having a Wnt/Frizzled-related disease, wherein the subject is identified by:
i) determining the level of at least one protein involved in the Wnt/Frizzled signaling pathway in a sample from the subject; and
ii) comparing the level of the at least one protein in the sample to a standard level of the at least one protein;
wherein a difference between the levels of the at least one protein in the sample identifies the subject as having a Wnt/Frizzled-related disease; and
b) administering a niclosamide compound, or salt thereof, to the subject in an amount effective to treat the disease,
wherein the Wnt/Frizzled-related disease is not a neoplasm.

4. The method of claim 3, wherein the Wnt/Frizzled-related disease is a cardiovascular disease.

5. A method of identifying a subject having a disease that is susceptible to treatment with a niclosamide compound, comprising:
a) determining the level of at least one protein involved in the Wnt/Frizzled signaling pathway in a sample from the subject; and
b) comparing the level of the at least one protein in the sample to a standard level of the at least one protein;
wherein a difference between the levels of the at least one protein in the sample identifies the subject as having a disease that is susceptible to treatment with a niclosamide compound.

6. A method of optimizing therapeutic efficacy for treatment of a Wnt/Frizzled-related disease, comprising:
a) determining the level of at least one protein involved in the Wnt/Frizzled signaling pathway in a sample from a subject having Wnt/Frizzled-related disease;
b) administering a niclosamide compound to the subject; and
c) subsequent to the administering of the niclosamide compound, determining the level of the at least one protein involved in the Wnt/Frizzled signaling pathway in the subject;
wherein when the level of the at least one protein is decreased by about 30% or less after administering the niclosamide compound, that level indicates a need to increase the amount of the niclosamide compound subsequently administered to the subject.

7. A method of predicting responsiveness of a cancer cell to treatment with a niclosamide compound, comprising:
a) determining the level of at least one protein involved in the Wnt/Frizzled signaling pathway in the cancer cell; and
b) comparing the level of the at least one protein to a standard level of the at least one protein;
wherein a difference between the levels of the at least one protein in the cancer cell indicates that the cancer cell is responsive to treatment with the niclosamide compound.

8. A method of detecting a neoplasm in a subject, comprising:
a) determining the level of at least one protein in a sample from the subject, wherein the protein is involved in the Wnt/Frizzled signaling pathway; and
b) comparing the level of the at least one protein to a standard level, wherein a difference between the levels of the at least one is indicative of the subject having a neoplasm.

9. The method of claim 5, wherein the level of the at least one protein is determined by determining the expression level of the mRNA encoding the protein.

10. The method of claim 5, wherein the at least one protein is selected from the group consisting of cytosolic β-catenin, a Wnt protein, Frizzled, and Dishevelled.

11. The method of claim 10, wherein the Wnt protein is Wnt3A.

12. The method of claim 5, wherein the Wnt/Frizzled-related disease is selected from the group consisting of a neoplasm and cardiovascular disease.

13. The method of claim 5, wherein the Wnt/Frizzled-related disease is a neoplasm.

14. The method of claim 13, wherein the neoplasm is cancer.

15. The method of claim 14, wherein the cancer is a carcinoma, an adenoma, a melanoma, a sarcoma, a lymphoma, a myeloid leukemia, a lymphatic leukemia, a blastoma, a glioma, an astrocytoma, a mesothelioma, or a germ cell tumor.

16. The method of claim 14, wherein the cancer is from colon, rectum, cervix, skin, epithelium, muscle, kidney, liver, lymph, bone, blood, ovary, prostate, lung, brain, or breast.

17. The method of claim 7, wherein the cancer cell is from a carcinoma, an adenoma, a melanoma, a sarcoma, a lymphoma, a myeloid leukemia, a lymphatic leukemia, a blastoma, a glioma, an astrocytoma, a mesothelioma, or a germ cell tumor.

18. The method of claim 7, wherein the cancer cell is from colon, rectum, cervix, skin, epithelium, muscle, kidney, liver, lymph, bone, blood, ovary, prostate, lung, brain, or breast.

19. A method of treating a Wnt/Frizzled-related disease in a subject in need of such treatment, the method comprising administering to the subject an effective amount of a niclosamide compound, or pharmaceutically acceptable salt thereof, of Formula I: 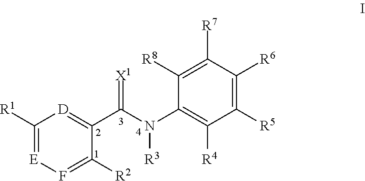
wherein
D is N or CR⁹;
E is N or CR¹⁰;
F is N or CR¹¹;
R¹ is H, halide, OR¹², SR¹³NR¹⁴R¹⁵, or described by one of the formulas: 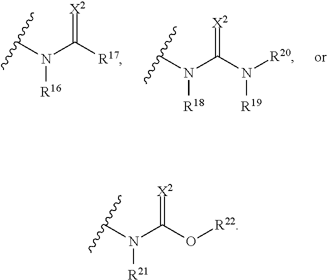
R² is H, OH, or OR¹²;
R³ is H, C₁₋₇ alkyl, C₂₋₇ alkenyl, C₂₋₇ alkynyl, C₂₋₆ heterocyclyl, C₆₋₁₂ aryl, C₇₋₁₄ alkylaryl, C₃₋₁₀alkylheterocyclyl, or C₁₋₇ heteroalkyl;
or R² and R³ combine to form a six-membered ring in which position 1 is connected to position 4 by one of the groups: 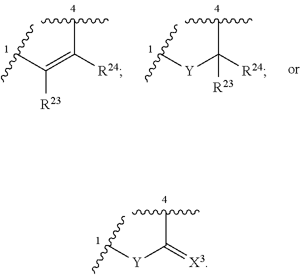
R⁴ and R⁸ are each, independently, selected from H, halide, CF₃, OR²⁸, C₁₋₇ alkyl, C₂₋₇ alkenyl, C₂₋₇ alkynyl, C₂₋₆ heterocyclyl, C₇₋₁₄ alkaryl, C₃₋₁₀ alkheterocyclyl, or C₁₋₇ heteroalkyl;
R⁵, R⁶, and R⁷ are each, independently, selected from H, C₁₋₇ alkyl, C₂₋₇ alkenyl, C₂₋₇ alkynyl, C₂₋₆ heterocyclyl, C₆₋₁₂ aryl, C₇₋₁₄ alkaryl, C₃₋₁₀ alkheterocyclyl, or C₁₋₇ heteroalkyl, halide, NO₂, CO₂H, SO₃H, CF₃, CN, OR²⁹, SR³⁰, or are described by the formulas: 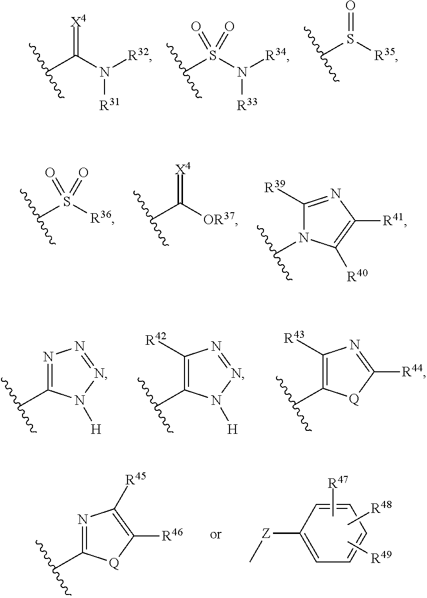
X¹, X², X³, and X⁴ is, independently, O, S; or NR³⁸;
Y is CR²⁵R²⁶, O, S, or NR²⁷;
Z is O, S, or CR⁵⁰R⁵¹;
Q is, independently, O, S, or NR⁵²;
R⁹, R¹⁰, and R¹¹ are each, independently, H, OH, OR¹², C₁₋₇ alkyl, C₂₋₇ alkenyl, C₂₋₇ alkynyl, C₁₋₇ heteroalkyl, halide, or NO₂;
R¹² and R¹³ are each, independently, acyl, C₁₋₇ alkyl, C₂₋₇ alkenyl, C₂₋₇-alkynyl, C₂₋₆ heterocyclyl, C₆₋₁₂ aryl, C₇₋₁₄ alkaryl, C₃₋₁₀ alkheterocyclyl, or C₁₋₇ heteroalkyl;
R¹⁷, R²², R³⁵, R³⁶, R³⁷, R³⁸ and R⁵² are each, independently, C₁₋₇ alkyl, C₂₋₇ alkenyl, C₂₋₇ alkynyl, C₂₋₆ heterocyclyl, C₆₋₁₂ aryl, C₇₋₁₄ alkaryl, C₃₋₁₀ alkheterocyclyl, or C₁₋₇ heteroalkyl;
R¹⁴, R¹⁵, R¹⁶, R¹⁸, R¹⁹, R²⁰, R²¹, R²³, R²⁴, R²⁵, R²⁶, R²⁷, R²⁸, R²⁹, R³⁰, R³¹, R³², R³³, R³⁴, and R⁴⁷ are each, independently, H, C₁₋₇ alkyl, C₂₋₇ alkenyl, C₂₋₇alkynyl, C₂₋₆ heterocyclyl, C₆₋₁₂ aryl, C₇₋₁₄ alkaryl, C₃₋₁₀ alkheterocyclyl, or C₁₋₇ heteroalkyl; and
R³⁹, R⁴⁰, R⁴¹, R⁴², R⁴³, R⁴⁴, R⁴⁵, R⁴⁶, R⁴⁷, R⁴⁸, R⁴⁹, R⁵⁰, and R⁵¹ are each, independently, H, halide, CN, NO₂, CF₃, C₁₋₇ alkyl, C₂₋₇ alkenyl, C₂₋₇ alkynyl, C₂₋₆ heterocyclyl, C₆₋₁₂ aryl, C₇₋₁₄ alkaryl, C₃₋₁₀ alkheterocyclyl, or C₁₋₇ heteroalkyl,
wherein the Wnt/Frizzled-related disease is cardiovascular disease.


SEQUENCE LISTING (6 sequences)

SEQ ID NO: 1 - protein, 352 aa, Homo sapiens
MAPLGYFLLLCSLKQALGSYPIWWSLAVGPQYSSLGSQPILCASIPGLVPKQLRFCRNYVEIMPSVAEGIKIGIQECQHQFRGRRWNCTTVHDSLAIFGPVLDKATRESAFVHAIASAGVAFAVTRSCAEGTAAICGCSSRHQGSPGKGWKWGGCSEDIEFGGMVSREFADARENRPDARSAMNRHNNEAGRQAIASHMHLKCKCHGLSGSCEVKTCWWSQPDFRAIGDFLKDKYDSASEMVVEKHRESRGWVETLRPRYTYFKVPTERDLVYYEASPNFCEPNPETGSFGTRDRTCNVSSHGIDGCDLLCCGRGHNARAERRREKCRCVFHWCCYVSCQECTRVYDVHTCK

SEQ ID NO: 2 - protein, 781 aa, Homo sapiens
MATQADLMELDMAMEPDRKAAVSHWQQQSYLDSGIHSGATTTAPSLSGKGNPEEEDVDTSQVLYEWEQGFSQSFTQEQVADIDGQYAMTRAQRVRAAMFPETLDEGMQIPSTQFDAAHPTNVQRLAEPSQMLKHAVVNLINYQDDAELATRAIPELTKLLNDEDQVVVNKAAVMVHQLSKKEASRHAIMRSPQMVSAIVRTMQNTNDVETARCTAGTLHNLSHHREGLLAIFKSGGIPALVKMLGSPVDSVLFYAITTLHNLLLHQEGAKMAVRLAGGLQKMVALLNKTNVKFLAITTDCLQILAYGNQESKLIILASGGPQALVNIMRTYTYEKLLWTTSRVLKVLSVCSSNKPAIVEAGGMQALGLHLTDPSQRLVQNCLWTLRNLSDAATKQEGMEGLLGTLVQLLGSDDINVVTCAAGILSNLTCNNYKNKMMVCQVGGIEALVRTVLRAGDREDITEPAICALRHLTSRHQEAEMAQNAVRLHYGLPVVVKLLHPPSHWPLIKATVGLIRNLALCPANHAPLREQGAIPRLVQLLVRAHQDTQRRTSMGGTQQQFVEGVRMEEIVEGCTGALHILARDVHNRIVIRGLNTIPLFVQLLYSPIENIQRVAAGVLCELAQDKEAAEAIEAEGATAPLTELLHSRNEGVATYAAAVLFRMSEDKPQDYKKRLSVELTSSLFRTEPMAWNETADLGLDIGAQGEPLGYRQDDPSYRSFHSGGYGQDALGMDPMMEHEMGGHHPGADYPVDGLPDLGHAQDLMDGLPPGDSNQLAWFDTDL

SEQ ID NO: 3 - protein, 647 aa, Homo sapiens
MAEEEAPKKSRAAGGGASWELCAGALSARLAEEGSGDAGGRRRPPVDPRRLARQLLLLLWLLEAPLLLGVRAQAAGQGPGQGPGPGQQPPPPPQQQQSGQQYNGERGISVPDHGYCQPISIPLCTDIAYNQTIMPNLLGHTNQEDAGLEVHQFYPLVKVQCSAELKFFLCSMYAPVCTVLEQALPPCRSLCERARQGCEALMNKFGFQWPDTLKCEKFPVHGAGELCVGQNTSDKGTPTPSLLPEFWTSNPQHGGGGHRGGFPGGAGASERGKFSCPRALKVPSYLNYHFLGEKDCGAPCEPTKVYGLMYFGPEELRFSRTWIGIWSVLCCASTLFTVLTYLVDMRRFSYPERPIIFLSGCYTAVAVAYIAGFLLEDRVVCNDKFAEDGARTVAQGTKKEGCTILFMMLYFFSMASSIWWVILSLTWFLAAGMKWGHEAIEANSQYFHLAAWAVPAIKTITILALGQVDGDVLSGVCFVGLNNVDALRGFVLAPLFVYLFIGTSFLLAGFVSLFRIRTIMKHDGTKTEKLEKLMVRIGVFSVLYTVPATIVIACYFYEQAFRDQWERSWVAQSCKSYAIPCPHLQAGGGAPPHPPMSPDFTVFMIKYLMTLIVGITSGFWIWSGKTLNSWRKFYTRLTNSKQGETTV

SEQ ID NO: 4 - protein, 670 aa, Homo sapiens
MAETKIIYHMDEEETPYLVKLPVAPERVTLADFKNVLSNRPVHAYKFFFKSMDQDFGVVKEEIFDDNAKLPCFNGRVVSWLVLAEGAHSDAGSQGTDSHTDLPPPLERTGGIGDSRPPSFHPNVASSRDGMDNETGTESMVSHRRERARRRNREEAARTNGHPRGDRRRDVGLPPDSASTALSSELESSSFVDSDEDGSTSRLSSSTEQSTSSRLIRKHKRRRRKQRLRQADRASSFSSITDSTMSLNIVTVTLNMERHHFLGISIVGQSNDRGDGGIYIGSIMKGGAVAADGRIEPGDMLLQVNDVNFENMSNDDAVRVLREIVSQTGPISLTVAKCWDPTPRSYFTVPRADPVRPIDPAAWLSHTAALTGALPRYELEEAPLTVKSDMSAVVRVMQLPDSGLEIRDRMWLKITIANAVIGADVVDWLYTHVEGFKERREARKYASSLLKHGFLRHTVNKITFSEQCYYVFGDLCSNLATLNLNSGSSGTSDQDTLAPLPHPAAPWPLGQGYPYQYPGPPPCFPPAYQDPGFSYGSGSTGSQQSEGSKSSGSTRSSRRAPGREKERRAAGAGGSGSESDHTAPSGVGSSWRERPAGQLSRGSSPRSQASATAPGLPPPHPTTKAYTVVGGPPGGPPVRELAAVPPELTGSRQSFQKAMGNPCEFFVDIM

SEQ ID NO: 5 - protein, 736 aa, Homo sapiens
MAGSSTGGGGVGETKVIYHLDEEETPYLVKIPVPAERITLGDFKSVLQRPAGAKYFFKSMDQDFGVVKEEISDDNARLPCFNGRVVSWLVSSDNPQPEMAPPVHEPRAELAPPAPPLPPLPPERTSGIGDSRPPSFHPNVSSSHENLEPETETESVVSLRRERPRRRDSSEHGAGGHRTGGPSRLERHLAGYESSSTLMTSELESTSLGDSDEEDTMSRFSSSTEQSSASRLLKRHRRRRKQRPPRLERTSSFSSVTDSTMSLNIITVTLNMEKYNFLGISIVGQSNERGDGGIYIGSIMKGGAVAADGRIEPGDMLLQVNDMNFENMSNDDAVRVLRDIVHKPGPIVLTVAKCWDPSPQAYFTLPRNEPIQPIDPAAWVSHSAALTGTFPAYPGSSSMSTITSGSSLPDGCEGRGLSVHTDMASVTKAMAAPESGLEVRDRMWLKITIPNAFLGSDVVDWLYHHVEGFPERREARKYASGLLKAGLIRHTVNKITFSEQCYYVFGDLSGGCESYLVNLSLNDNDGSSGASDQDTLAPLPGATPWPLLPTFSYQYPAPHPYSPQPPPYHELSSYTYGGGSASSQHSEGSRSSGSTRSDGGAGRTGRPEERAPESKSGSGSESEPSSRGGSLRRGGEASGTSDGGPPPSRGSTGGAPNLRAHPGLHPYGPPPGMALPYNPMMVVMMPPPPPPVPPAVQPPGAPPVRDLGSVPPELTASRQSFHMAMGNPSEFFVDVM

SEQ ID NO: 6 - protein, 716 aa, Homo sapiens
MGETKIIYHLDGQETPYLVKLPLPAERVTLADFKGVLQRPSYKFFFKSMDDDFGVVKEEISDDNAKLPCFNGRVVSWLVSAEGSHPDPAPFCADNPSELPPPMERTGGIGDSRPPSFHPHAGGGSQENLDNDTETDSLVSAQRERPRRRDGPEHATRLNGTAKGERRREPGGYDSSSTLMSSELETTSFFDSDEDDSTSRFSSSTEQSSASRLMRRHKRRRRKQKVSRIERSSSFSSITDSTMSLNIITVTLNMEKYNFLGISIVGQSNERGDGGIYIGSIMKGGAVAADGRIEPGDMLLQVNEINFENMSNDDAVRVLREIVHKPGPITLTVAKCWDPSPRGCFTLPRSEPIRPIDPAAWVSHTAAMTGTFPAYGMSPSLSTITSTSSSITSSIPDTERLDDFHLSIHSDMAAIVKAMASPESGLEVRDRMWLKITIPNAFIGSDVVDWLYHNVEGFTDRREARKYASNLLKAGFIRHTVNKITFSEQCYYIFGDLCGNMANLSLHDHDGSSGASDQDTLAPLPHPGAAPWPMAFPYQYPPPPHPYNPHPGFPELGYSYGGGSASSQHSEGSRSSGSNRSGSDRRKEKDPKAGDSKSGGSGSESDHTTRSSLRGPRERAPSERSGPAASEHSHRSHHSLASSLRSHHTHPSYGPPGVPPLYGPPMLMMPPPPAAMGPPGAPPGRDLASVPPELTASRQSFRMAMGNPSEFFVDVM